(19) United States
(12) Patent Application Publication
(10) Pub. No.: US 2003/0187317 A1
(43) Pub. Date: Oct. 2, 2003

(54) Acid and other oxygenate reduction in an olefin containing feed stream

(75) Inventors: Petra De Wet, Vanderbijlpark (ZA); Hubert Naude, Johannesburg (ZA); Deirdre Diamond, Johannesburg (ZA)

Correspondence Address: KNOBBE, MARTENS, OLSON AND BEAR LLP, SUITE 1600, 620 NEWPORT CENTER DRIVE, NEWPORT BEACH, CA, 92660, US

(73) Assignee: SASOL TECHNOLOGY (PTY) LTD., JOHANNESBURG (ZA)

(21) Appl. No.: 09/957,445

(22) Filed: Sep. 19, 2001

(51) Int. Cl. C07C 7/00

(52) U.S. Cl. 585/864; 585/809

(57) ABSTRACT

The invention relates to the reduction of oxygenates, including acid, from an olefin containing feedstream. Typically the feedstream is of Fischer-Tropsch process origin and includes hydrocarbons, such as olefins, paraffins, and aromatics, as well as oxygenates, including acid.

### FIELD OF THE INVENTION

The invention relates to the reduction of oxygenates, including acid, from an olefin containing feedstream. Typically the feedstream is of Fischer-Tropsch process origin and includes hydrocarbons, such as olefins, paraffins, and aromatics, as well as oxygenates, including acid.

### BACKGROUND TO THE INVENTION

In the production of olefins, products such as 1-octene, oxygenates, including acid, are undesirable components and need to be reduced or completely removed in order to produce a commercially acceptable product.

At present it is known to remove or reduce the oxygenate, including acid, by using a process as described below.

Existing Technology:

The octene train makes use of a potassium carbonate wash to remove acids from the feed. The carbonate is regenerated in a closed loop process, which involves the incineration of the potassium organic salts formed in the wash unit. The acid-free feed then undergoes pre-fractionation to remove lights and heavies and is then referred to as a C₈ broadcut. The next processing step is oxygenate removal which is an extractive distillation with NMP to remove oxygenates such as ketones and aldehydes.

Acid Removal and Oxygenate Removal thus occur in two separate processing steps.

The above technology is sensitive to the design acid number of the feed stream.

### SUMMARY OF THE INVENTION

According to a first aspect of the invention, there is provided a process for the reduction of oxygenates, including acid, in an olefin and paraffin containing hydrocarbon feed stream, said process including azeotropic distillation of the feed stream using a binary entrainer to recover at least the olefin and paraffin portion of the feed stream.

The binary entrainer may include a polar species.

The polar species may be acetonitrile.

The binary entrainer may include a solvent, such as an alcohol, which is also a polar species.

The binary entrainer may include water.

The feed stream is typically of Fischer Tropsch process origin containing hydrocarbons, such as olefins and/or paraffins and/or aromatics, and impurities, such as acid and other oxygenates.

The feed stream may include C₇ to C₁₂ hydrocarbons of olefinic and paraffinic nature.

The feed stream may be fed to the azeotropic distillation column at an intermediate feed point.

The azeotropic disitillation column reflux may be a recycle stream that contains a mixture of binary entrainer and olefin enriched hydrocarbons.

The hydrocarbons in the feed stream may form an azeotrope with the binary entrainer in order to recover the ternary acids-and-other-oxygenate-impoverished-hydrocarbon-binary-entrainer azeotrope overhead from the azeotropic distillation column.

Acids and other oxygenates may be recovered from the bottoms of this column. In one embodiment, virtually all the acids and other oxygenates are recovered from said bottoms.

The binary entrainer may be a mixture of ethanol and water. However, alternative solvents of the binary entrainer include one or more of methanol, propanol, iso-propanol, butanol, and acetonitrile.

The distillate from the azeotropic distillation column may be condensed and sub-cooled, optionally, together with an overheads stream from an associated stripper column.

The condensed stream may then be routed to a phase separator where a light hydrocarbon-rich phase is separated from a heavier solvent-rich phase.

The heavy phase which consists mainly of the binary entrainer components i.e. solvent and water, and also hydrocarbon species, may be routed to the azeotropic distillation column as binary entrainer.

The light phase may be mainly acid and other oxygenate impoverished or free hydrocarbon material with some solvent of binary entrainer origin, and very little water.

The light phase may be fed to the associated stripper column where the acid and oxygenate free hydrocarbons are recovered in the bottoms. The overhead vapour product from this column is a solvent-hydrocarbon azeotrope, which may be returned to the overheads condenser.

Without being bound by theory, it is believed that the binary entrainer results in the formation of a ternary azeotrope, which is the dominant distillate product of the azeotropic distillation column.

It is believed that the polar species of the binary entrainer forms the low-boiling binary azeotrope with the non-oxygenate portion of the feed stream but not with the acid and other oxygenate portion thereof.

The azeotrope may be homogeneous or heterogeneous depending on the choice of binary entrainer, polar species or solvent.

The addition of water enhances phase separation in all instances. However, where the azeotrope is homogeneous, the addition of water results in phase separation being possible.

Addition of water also results in the formation of a low-boiling ternary azeotrope, which is richer in hydrocarbon (non-oxygen containing species) content, thus improving the efficiency of the azeotropic distillation process.

It is one advantage of the invention that the addition of water to the solvent or polar species to form the binary entrainer results in the formation of a heterogeneous ternary azeotrope, and so facilitates phase separation of the distillate. The solvent phase can be recovered in a phase separator instead of another separation process. (If the binary hydrocarbon-solvent azeotrope is pressure-sensitive, distillation can be used to recover the solvent. This is more energy intensive than phase separation.)

A further advantage of the ternary azeotrope used to recover the hydrocarbons by reduction of the acids and other oxygenates is that this azeotrope is richer in hydrocarbons than the binary solvent-hydrocarbon azeotrope. Considerably less solvent and energy is required to recover the hydrocarbons to the distillate of the azeotropic column.

Yet a further advantage is that this choice of solvent results in an environmentally friendly process when compared with other solvent options.

Yet a further advantage is that this process is more environmentally friendly than currently used carbonate wash and incineration processes. The choice of an environmentally friendly solvent, such as ethanol, can further enhance the environmentally friendly qualities of this process.

Yet a further advantage is that the azeotropic distillation process is robust in terms of feed acid content.

### BRIEF DESCRIPTION OF AN EMBODIMENT OF THE INVENTION

What follows are two examples of the removal of acid and other oxygenates from a C₇ to C₁₂ olefin containing feed stream i.e. a C₈ broadcut of a Fischer-Tropsch process. In the first example the acid and other oxygenates are removed with the aid of an azeotropic distillation using acetonitrile and water as binary entrainer and in the second example using ethanol and water as the binary entrainer.

The examples are by way of illustration only and are in no way limiting of the broad principles of the invention.

### EXAMPLE 1

### Acid and Other Oxygenate Removal from Ca Broadcut Using Azeotropic Distillation with Acetonitrile.

An azeotropic distillation process to remove acids and oxygenates from C₈ broadcut using acetonitrile as the solvent was piloted in glass columns. It was aimed to firstly prove the process concept, and secondly to collect at least two sets of data point samples for the stripper and azeotropic columns, under stable operating conditions. The process was piloted without closing the solvent loop.

Aspen™ simulations have been able to closely approximate the results obtained on the pilot plant. The predicted product stream composition and column profile temperature results match the experimental data well.

From the pilot plant experimental work it appears that the 1-octene recovery may exceed 98.5%. The hexanal specification is met in the stripper bottoms, and the acetonitrile levels in the azeotropic and stripper column bottoms are within specification.

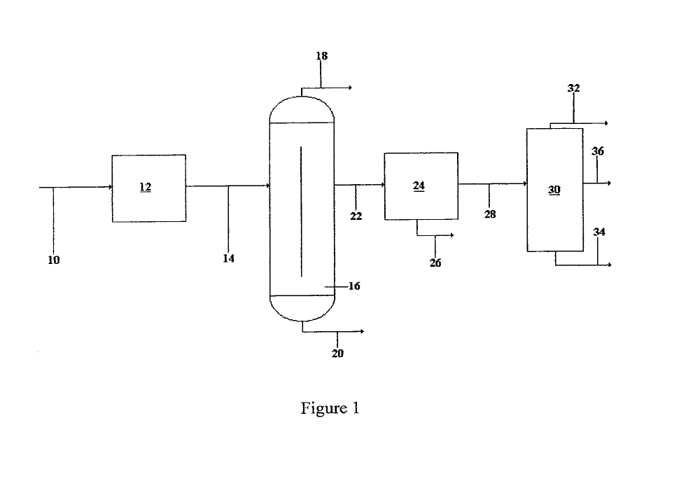

A conventional 1-octene plant, as shown in FIG. 1, includes 3 basic steps:

1. Organic acid removal using a potassium carbonate wash.

2. Oxygenate extraction using extractive distillation with NMP.

3. Super-fractionation to produce co-monomer grade 1-octene.

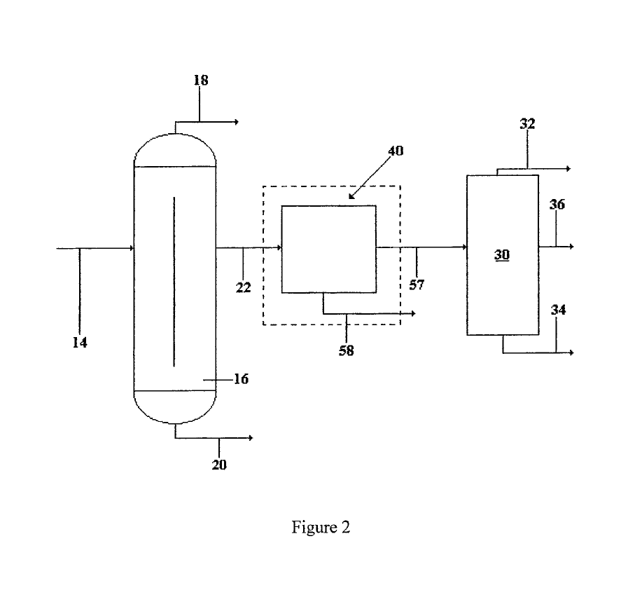

In the present invention, as embodied in this example and as shown in FIG. 2, steps 1 and 2 can be combined in the acetonitrile azeotropic distillation process, after pre-fractionation. This necessitates a stainless steel pre-fractionator. The product from the azeotropic distillation process will be acid free C₈ broadcut, containing minimal oxygenates. This product can be super-fractionated.

In FIG. 1, an olefinic feed stream 10 is fed to a potassium carbonate wash 12 from which an acid free olefin stream 14 C₇-C₁₂ is fed to a splitter 16 made of carbon steel. The splitter 16 has 3 product streams, a C₇₋ overhead stream 18, a bottoms C₉₊ stream 20, and a C₈ broadcut stream 22 which is fed to NMP extractrive distillation 24 from which an oxygenate stream 26 is drawn of and a product 28 is fed to super fractionation 30 from which a C₈ lights stream 32, a C₈ heavies stream 34 and a co-monomer grade octane 36 is recovered.

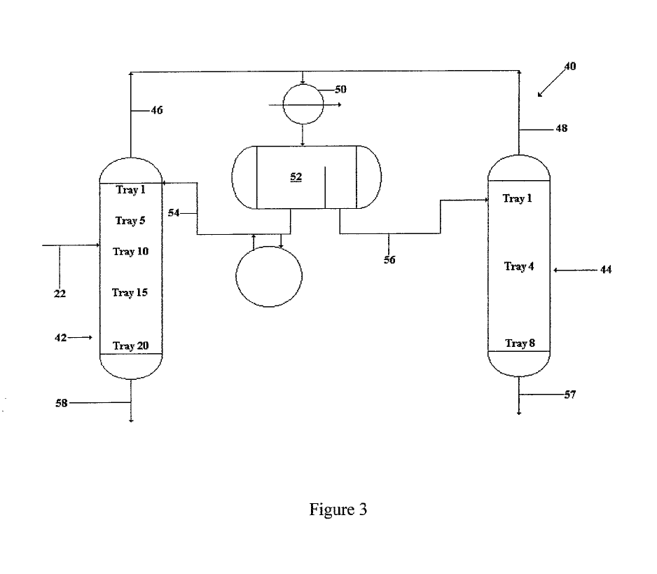

In FIGS. 2 and 3, the azeotropic distillation process 40 would make use of an azeotropic 42 and a stripper column 44. The overheads 46, 48 of both columns will report to a combined condenser 50 and reflux drum 52. The combined overhead streams phase separate on cooling. The acetonitrile phase 54 is recycled to the azeotropic column 42, and the hydrocarbon phase 57 is fed to the stripper column 44.

The process would require water removal from the solvent loop 46, 54. This is because the possibility exists that esterification reactions could cause a build-up of water in the solvent loop.

The azeotropic column bottoms product 58 consists of oxygenates and acids, and the stripper column bottoms 57 is the hydrocarbon product stream.

Azeotropic Column 42

A 50-mm diameter glass Oldershaw column for aqueous systems with 40 actual trays was used. The feed 22 reported to tray 21, and the reflux 54 to tray 1 (top of column). Distillate was collected in the phase separator 52. The heavy solvent phase 54 from the phase separator was recycled to the azeotropic column as reflux.

Stripper Column 44

A 50-mm diameter glass Oldershaw column for organic systems with 20 trays was used. The feed 56 for this column reported to tray 1. The distillate 48 reported to the phase separator 52. The light hydrocarbon phase 56 from the phase separator was the feed for this column.

Phase Separator 52

A jacketed glass phase separator was used. The operating temperature of the phase separator was effectively controlled at 45° C. by means of a Lauda Bath.

It is expected that a commercial plant would require a pervaporation unit 58 or distillation column to control the water content of the solvent recycle 54 to the azeotropic column 42. During piloting, the water content was controlled by addition of dry acetonitrile, and thus the solvent cycle was not closed.

Analyses of product streams were done by GC-FID on a FFAP (polar) column. The acetonitrile content of the azeotropic 42 and stripper column 44 was determined on a PONA (non-polar) column. Water content analyses were done by means of Karl Fischer.

Data Logging:

Spot checks were made of all flow rates on an hourly basis, and logged.

2-minute averages of the azeotropic column profile and feed temperatures were logged via a PLC system.

All other temperatures were manually logged on the hour every hour.

Five sets of data point samples were taken. All samples were analyzed, all flows and temperatures were plotted and mass balances were calculated. All of this information was evaluated before a decision was taken whether the plant was stable for a long enough period when the samples were drawn, to warrant further processing of data, whereafter two data points were selected at which the plant was stable, namely points 4 and 5.

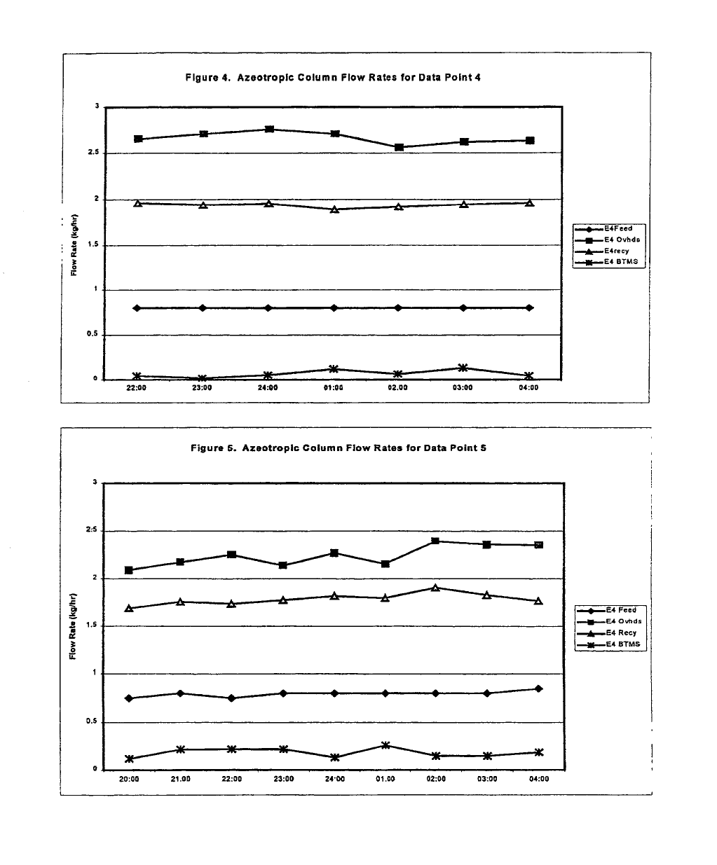

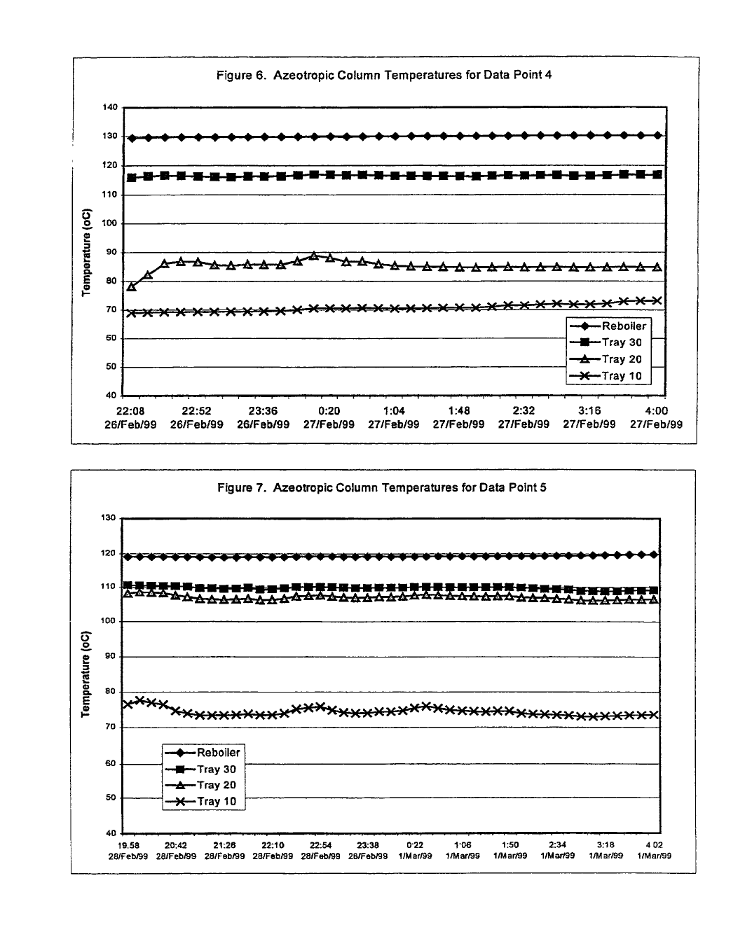

Graphical representations of constant feed and product flows as well as constant profile temperatures for data points 4 and 5 are shown in FIGS. 4, 5, 6 and 7 and shown in tables 1, 2 and 3.

Constant analytical results for critical components in product steams.

TABLE 1
Acetonitrile Content of Azeotropic Column Bottoms

| | |
|---|---|
| Data Point 4 | Data Point 5 |

| Time | Concentration (ppm) | Time | Concentration (ppm) |
|---|---|---|---|
| 00:00 | 9.1 | 20:00 | 74.9 |
| 02:00 | 23.8 | 22:00 | 64.6 |
| 04:00 | 12.2 | 00:00 | 48.3 |
| 06:00 | 0.0 | 04:00 | 13.6 |

The acetonitrile content for the 8 hours preceding data point 4 was stable at low concentrations. For the 8 hours preceding data point 5, the acetonitrile content decreased constantly as the bottoms stream approached ‘on-specification’ status.

Similar analytical results for the phases from the phase separator and those of the two recycle containers.

TABLE 2
Compositions of Azeotropic Column Solvent and Phase
Separator Heavy Phase for Data Point 4

| | 1- | n- | 2-Hexa- | | | |
|---|---|---|---|---|---|---|
| Stream | Octene | Octane | none | Hexanal | Water | Acetonitrile |
| Phase | 2.886 | 0.217 | 0.072 | 0.061 | 15.58 | 79.34 |
| Separator | | | | | | |
| Heavy | | | | | | |
| Phase | | | | | | |
| Azeo- | 2.332 | 0.172 | 0.047 | 0.025 | 15.36 | 79.76 |
| tropic | | | | | | |
| Column | | | | | | |
| Solvent | | | | | | |

TABLE 3
Compositions of Azeotropic Column Solvent and Phase
Separator Heavy Phase for Data Point 5

| | 1- | n- | 2-Hexa- | | | |
|---|---|---|---|---|---|---|
| Stream | Octene | Octane | none | Hexanal | Water | Acetonitrile |
| Phase | 2.800 | 0.211 | 0.040 | — | 14.48 | 80.08 |
| Separator | | | | | | |
| Heavy | | | | | | |
| Phase | | | | | | |
| Azeo- | 2.678 | 0.233 | 0.039 | — | 14.5 | 80.28 |
| tropic | | | | | | |
| Column | | | | | | |
| Solvent | | | | | | |

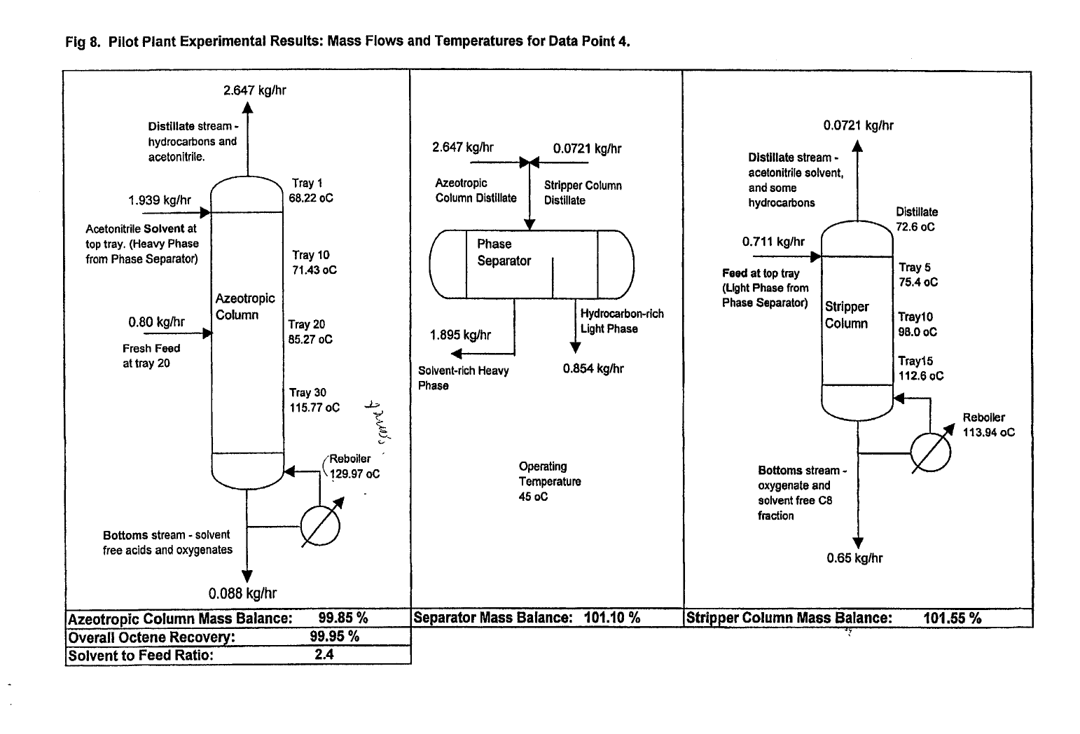

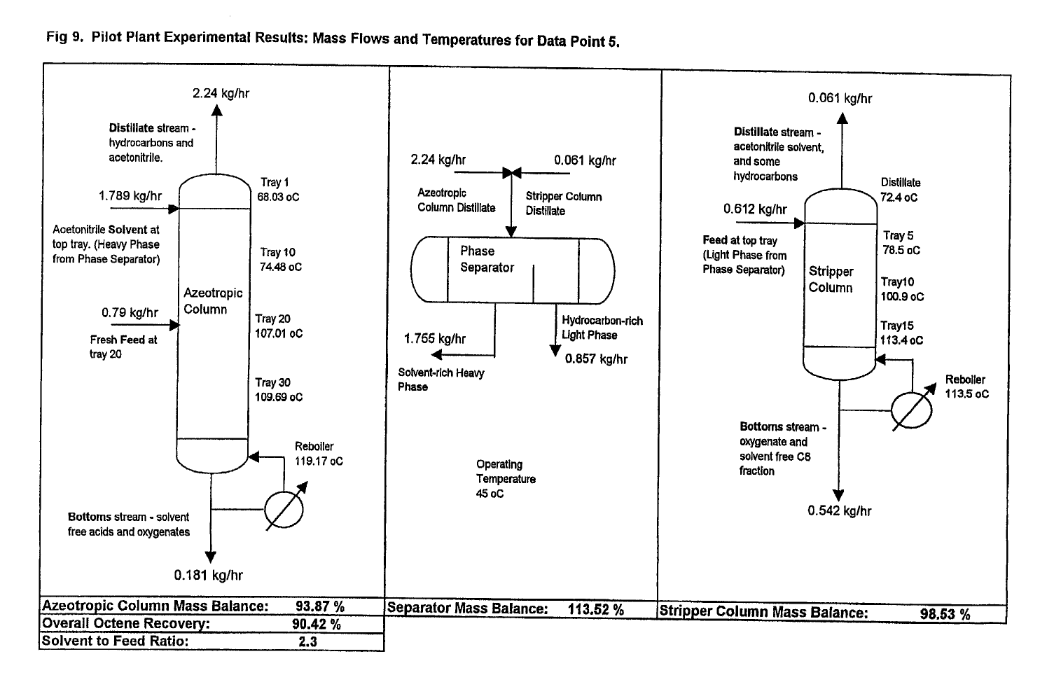

The measured mass flows and temperatures are presented in FIGS. 8 and 9. The octene recoveries are calculated by determining the ratio of octene in the stripper bottoms, to the total octene in both bottoms streams.

The solvent: feed ratio was higher for data point 4. It can also be seen from the temperature profiles, that the azeotropic column ran at higher bottoms temperature than for data point 5.

Critical Component Analytical Results for Data Point 4

The distillate samples for both the azeotropic and stripper columns phase separate as a result of cooling from process to ambient temperature. The results for both phases are presented here in tables 4, 5 and 6.

TABLE 4
Azeotropic Column (wt %)

| | 1- | n- | 2-Hexa- | | | |
|---|---|---|---|---|---|---|
| Stream | Octene | Octane | none | Hexanal | Water | Acetonitrile |
| Solvent | 2.332 | 0.172 | 0.047 | 0.025 | 15.36 | 79.760 |
| Distillate | 55.911 | 9.072 | 0.060 | 0.040 | 0.05 | 5.325 |
| Light | | | | | | |
| Phase | | | | | | |
| Distillate | 2.233 | 0.176 | 0.036 | 0.037 | 14.5 | 80.291 |
| Heavy | | | | | | |
| Phase | | | | | | |
| Bottoms | 0.203 | 0.039 | 41.429 | 2.628 | n.a. | 0.000 |

TABLE 5
Stripper Column (wt %)

| | 1- | n- | 2-Hexa- | | | |
|---|---|---|---|---|---|---|
| Stream | Octene | Octane | none | Hexanal | Water | Acetonitrile |
| Feed | 55.016 | 9.137 | 0.023 | 0.019 | 0.05 | 6.053 |
| Distillate | 46.930 | 9.281 | 0.014 | 0.014 | 0.03 | 9.349 |
| Light | | | | | | |
| Phase | | | | | | |
| Distillate | 7.448 | 0.884 | 0.061 | 0.025 | 1.02 | 84.030 |
| Heavy | | | | | | |
| Phase | | | | | | |
| Bottoms | 59.250 | 9.828 | — | — | n.a. | 0.000 |

TABLE 6
Phase Separator (wt %)

| | 1- | n- | 2-Hexa- | | | |
|---|---|---|---|---|---|---|
| Stream | Octene | Octane | none | Hexanal | Water | Acetonitrile |
| Heavy | 2.436 | 0.188 | 0.073 | 0.061 | 15.58 | 79.337 |
| Phase | | | | | | |
| Light | 54.935 | 9.079 | 0.05 | 0.036 | n.a. | 6.439 |
| Phase | | | | | | |

Critical Component Analytical Results for Data Point 5

The distillate samples for both the azeotropic and stripper columns phase separate as a result of cooling from process to ambient temperature. The results for both phases are presented here in tables 7, 8 and 9.

TABLE 7
Azeotropic Column (wt %)

| | 1- | n- | 2-Hexa- | | | |
|---|---|---|---|---|---|---|
| Stream | Octene | Octane | none | Hexanal | Water | Acetonitrile |
| Solvent | 2.637 | 0.196 | 0.039 | — | 14.96 | 79.904 |
| Distillate | 56.160 | 9.020 | — | — | n.a. | 5.553 |
| Light | | | | | | |
| Phase | | | | | | |
| Distillate | 2.678 | 0.233 | 0.039 | — | 14.5 | 80.277 |
| Heavy | | | | | | |
| Phase | | | | | | |
| Bottoms | 19.011 | 4.487 | 26.301 | 1.951 | n.a. | 0.0014 |

TABLE 8
Stripper Column (wt %)

| | 1- | n- | 2-Hexa- | | | |
|---|---|---|---|---|---|---|
| Stream | Octene | Octane | none | Hexanal | Water | Acetonitrile |
| Solvent | 56.160 | 9.020 | — | — | n.a. | 5.553 |
| Distillate | 45.846 | 9.000 | 0.043 | — | n.a. | 6.441 |
| Light | | | | | | |
| Phase | | | | | | |
| Distillate | 7.344 | 0.869 | 0.133 | — | 1.00 | 82.892 |
| Heavy | | | | | | |
| Phase | | | | | | |
| Bottoms | 60.250 | 9.370 | — | — | n.a. | 0.000 |

TABLE 9
Phase Separator (wt %)

| | 1- | n- | 2-Hexa- | | | |
|---|---|---|---|---|---|---|
| Stream | Octene | Octane | none | Hexanal | Water | Acetonitrile |
| Heavy | 2.800 | 0.211 | 0.040 | — | 14.48 | 80.087 |
| Phase | | | | | | |
| Light | 57.407 | 9.122 | — | — | n.a. | 5.094 |
| Phase | | | | | | |

Symbol: ‘-’, Status: undetected components on GC results

Symbol: ‘n.a.’, Status: no analysis done

Feed Composition

Using the GC-MS trace of a C₈ broadcut done on a polar column as basis, the most important hydrocarbons and all the oxygenate components were identified in the feed. The hydrocarbon fraction was converted to actual components by using the components and relative quantities as per the C₈ broadcut composition of a conventional process. Refer to the GC table, table 20.

Mass Balances and Product Compositions

Azeotropic Column 42:

The feed and reflux flow rates to the azeotropic column were measured on scales. The overheads flow was determined from volumetric and density measurements, while the bottoms flow was very dependent on the level in the reboiler. Therefore it was assumed that the azeotropic column reflux and feed flow rates were accurately determined.

Stripper Column 44:

Using the simulation results obtained for the azeotropic column as basis an overall plant mass balance was calculated. This fixed the stripper column bottoms flow. The number of theoretical stages was fixed at eight. The feed flow rate to the stripper was manipulated to match the bottoms 1-octene and n-octane experimental data.

A comparison between measured and simulated mass flow rates is presented in tables 10 and 11. The reconciled mass flow rates are optimized up to 5 decimal places in certain cases. This is because, at low flow rates, a change in a mass flow rate, even at the 5^{th} decimal, can result in substantial product composition changes.

TABLE 10
Mass flow Rates for Data Point 4

| | | Simulation |
|---|---|---|
| Stream | Measured (kg/hr) | (kg/hr) |
| Azeotropic Column Feed | 0.800 | 0.800 |
| Azeotropic Column Solvent | 1.939 | 1.939 |
| Azeotropic Column Distillate | 2.647 | 2.6210 |
| Azeotropic Column Bottoms | 0.088 | 0.1180 |
| Stripper Column Feed | 0.711 | 0.7720 |
| Stripper Column Distillate | 0.072 | 0.090 |
| Stripper Column Bottoms | 0.65 | 0.682 |
| Azeotropic Column Mass Balance | 99.85396 | 100.0 |
| Stripper Column Mass Balance | 101.5471 | 100.0 |
| Overall System Mass Balance | 92.25 | 100.0 |

TABLE 11
Mass flow Rates for Data Point 5

| | | Simulation |
|---|---|---|
| Stream | Measured (kg/hr) | (kg/hr) |
| Azeotropic Column Feed | 0.788 | 0.788 |
| Azeotropic Column Solvent | 1.789 | 1.789 |
| Azeotropic Column Distillate | 2.24 | 2.4118 |
| Azeotropic Column Bottoms | 0.181 | 0.16525 |
| Stripper Column Feed | 0.612 | 0.6950 |
| Stripper Column Distillate | 0.061 | 0.0723 |
| Stripper Column Bottoms | 0.542 | 0.6227 |
| Azeotropic Column Mass Balance | 93.87359 | 100.0 |
| Stripper Column Mass Balance | 98.52941 | 100.0 |
| Overall System Mass Balance | 91.51899 | 100.0 |

The material balances as shown in tables 10 and 11 were used for the simulations. The simulations were performed on Aspen Plus™ using the Unifac Dortmund group contribution method to predict the vapour-liquid and liquid-liquid equilibrium data. Tables 21 to 24 show the Aspen™ simulation for the azeotropic stripper columns for data points 4 and 5.

In FIG. 10, the azeotropic column temperature profile for data point 4 differs at the feed point—the feed entered at 105° C. The predicted profile for data point 5 matches the plant data well. These azeotropic column profiles are simulated for profile sampling conditions.

The stripper column profiles, simulated for data point conditions, differ from the measured data in the middle stages of the column. For Data Point 4, the stripper column had a hotter profile in the top stages, and for data point 5, the stripper column ran colder in the top stages.

For all columns, the simulation matches the measured distillate and bottoms product temperatures well.

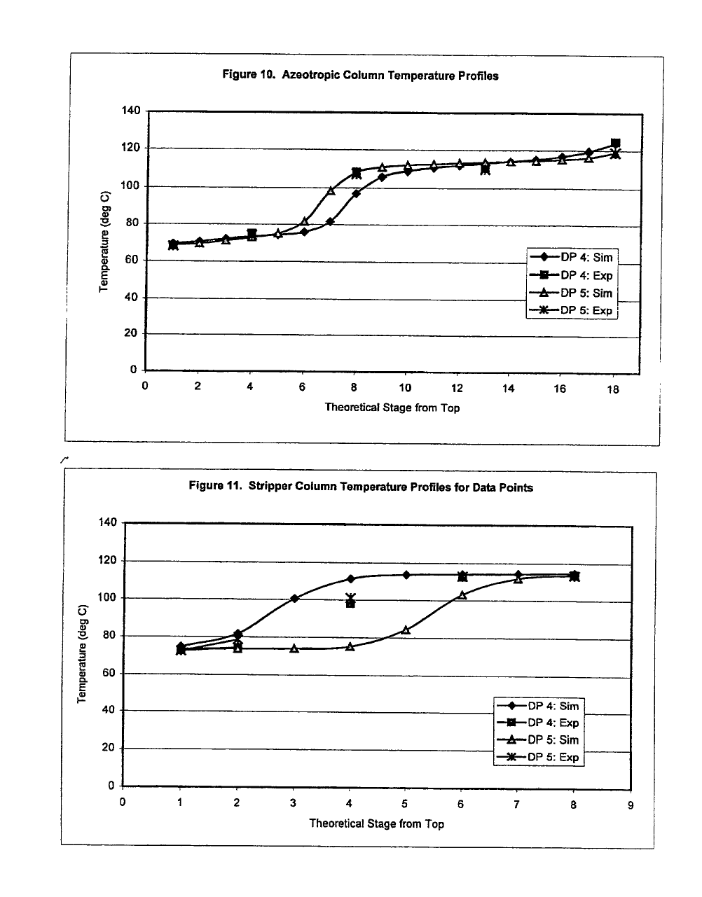

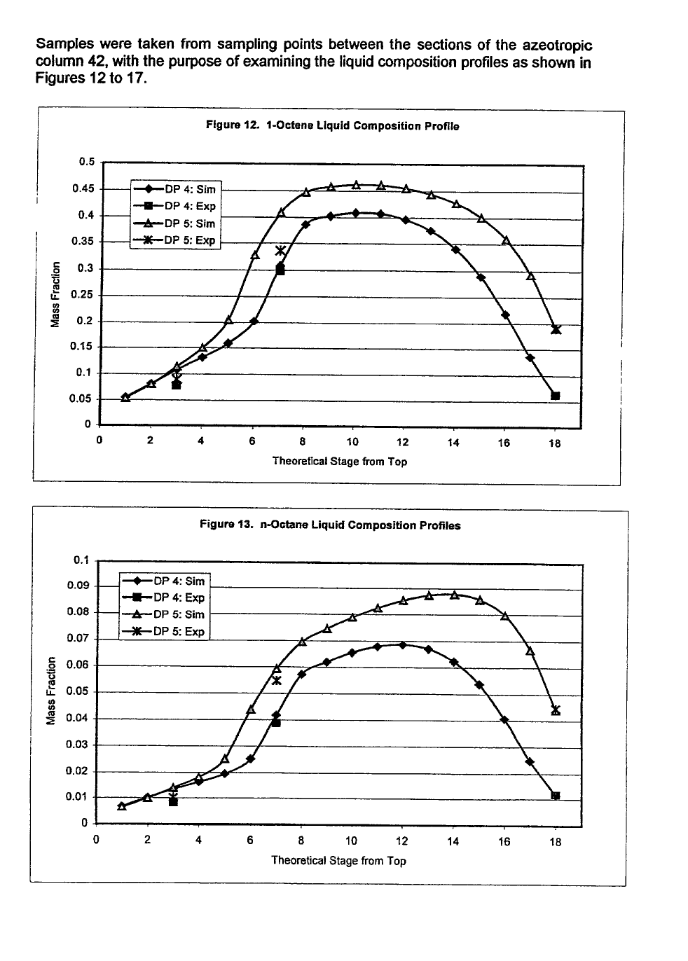

Samples were taken from sampling points between the sections of the azeotropic column 42, with the purpose of examining the liquid composition profiles as shown in FIGS. 12 to 17.

The profile samples for data point 4 were taken a day after the product data point samples. The average mass flow rates, and temperatures for the column had changed by this stage and the profiles were simulated at these new flow conditions. Recycle and bottoms samples were also taken. In the case of data point 5, the profile samples were taken a few hours after the data point samples, and no recycle or bottoms samples were taken. In this case the recycle and bottoms compositions of the data point were used for the simulation of the azeotropic column.

Profile samples could only be taken above the feed point. The sample points were located between column sections, and the liquid samples were of the tray above the sample point.

The simulations of both data point 4 and 5 profiles yielded the best results (in terms of 1-octene and n-octane bottoms concentration) for an azeotropic column with 18 stages, and the feed reporting to stage 8.

In the case of 1-octene profiles in FIG. 12, both the simulation and experimental results indicate a concentration bulge in the middle stages of the column. At lower stage numbers (stages near the top of the column), the simulation predicts a higher 1-octene presence than experimentally determined, for both data point profiles. The data point 4 simulation matches the experimental profile very well. There is good agreement for the product stream concentrations.

The profiles of n-octane in FIG. 13 bear strong resemblance to those of 1-octene for corresponding data points. The predicted and measured profiles, as well as product stream concentrations agree well.

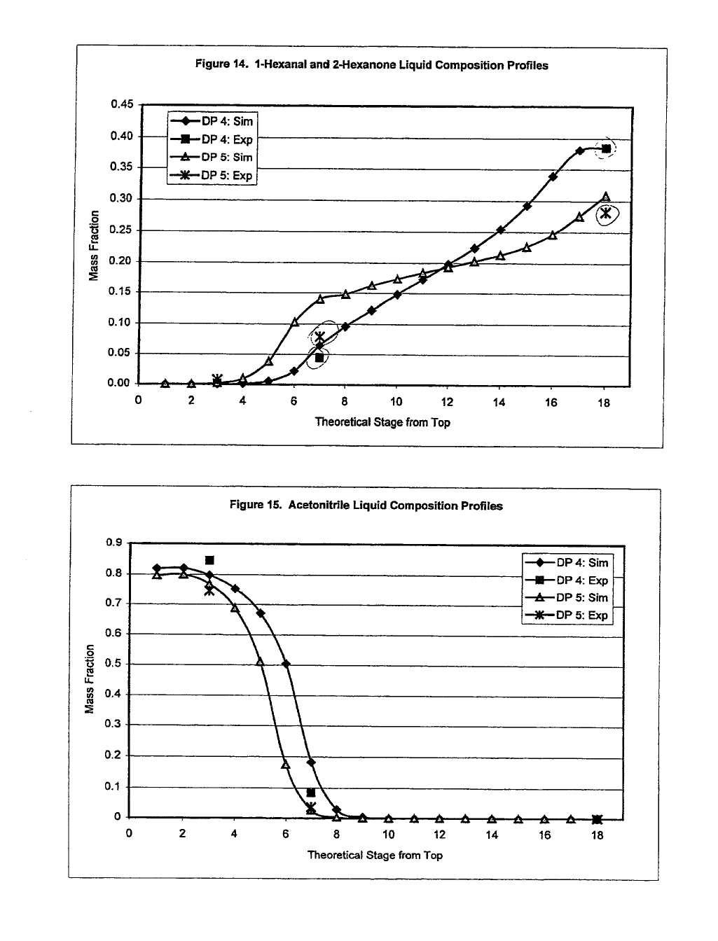

Combining the 2-hexanone and 1-hexanal concentrations compensates for integration errors that result because of their close proximity on the GC-traces. Referring to FIG. 14 for their concentration profiles, there is fairly good agreement between predicted and experimental data, especially for profile 4. The simulation predicts the significant increase in the measured concentrations of these components between stages 5 and 18.

Both the predicted and measured column profiles for acetonitrile in FIG. 15, reflect a sharply decreasing concentration profile from the top to the feed stages, and indicate that negligible amounts of acetonitrile are present below the feed stage.

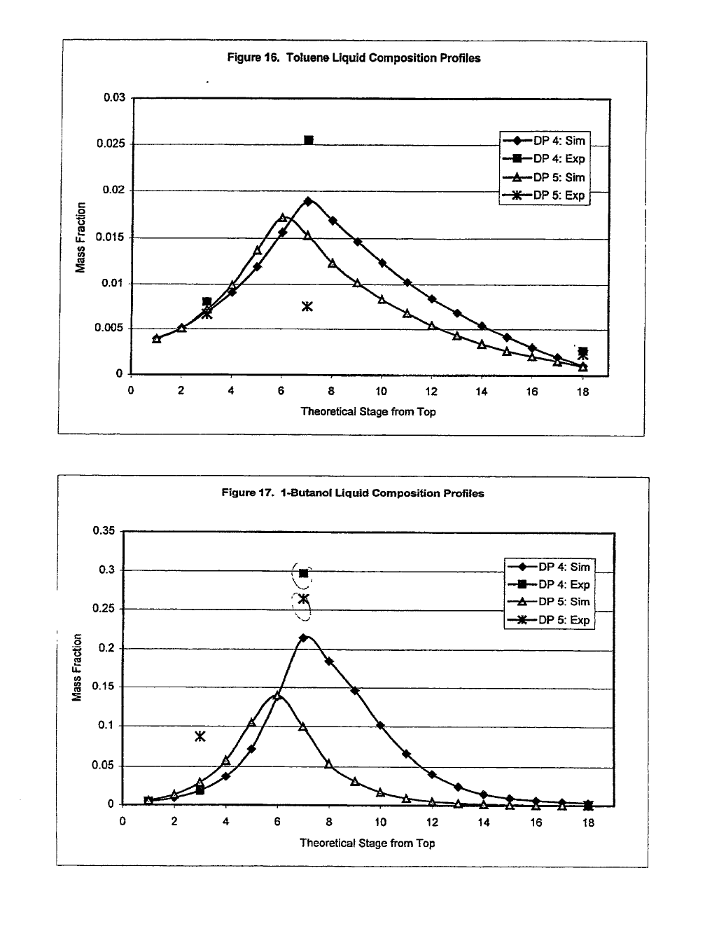

Both simulations predict a sharp toluene concentration peak between stages 5 and 11 (from the top). The experimental results for profile 4 indicate that a much higher toluene concentration in the azeotropic column than was predicted. The results for profile 5 indicate significantly lower toluene concentrations than was predicted. There is not a strong agreement between the simulated and experimental data as presented in FIG. 16.

The profile 4 and 5 simulations predict concentration peaks for 1-butanol in the middle stages of the column. The predicted concentrations are significantly lower than was determined experimentally, as can be seen in FIG. 17.

The same feed composition was used for both the data point 4 and data point 5 simulations. The GC-results for the solvent recycle to the azeotropic column, and the feed to the stripper column was used as input to the simulation. Manipulated column parameters include bottoms flow rates, and theoretical number of stages. For data point 4, the azeotropic column was simulated with 18 theoretical stages (feed at stage 8), and for data point 5, the azeotropic column was simulated with 19 theoretical stages (feed at stage 9).

Azeotropic Column Bottoms 58:

For both data points, there is a good match for the azeotropic column bottoms 1-octene and n-octane concentration results (tables 12 and 16). This is because column parameters were manipulated to obtain a good match for these two components. The corresponding predicted 2-hexanone concentration for data point 4 is also close to the experimental data for that design run. The presence of trace amounts of acetonitrile in the bottoms for data point 5 is not predicted by the simulation, which predicts no acetonitrile in this stream. The simulation also predicts higher concentrations of 2-hexanone and hexanal for data point 5, than was experimentally determined. The simulated and experimental values for both data points compare reasonably well for all the components.

Stripper Column Bottoms 56:

The measured and simulated data for the stripper column bottoms compares very well for both data points (tables 14 and 18). There is a good match for the 1-octene and n-octane concentration results. Once again column parameters were manipulated to obtain a good match for these two components.

Because the distillate samples of the two columns underwent phase separation, and the respective weights of the light and heavy phases were unknown, the distillate stream for these columns could not be directly compared with simulated data. In order to compare the plant and simulated results, the phase separation was simulated at low temperatures in Aspen™. The phase separation temperature was manipulated in an attempt to match plant and simulated data.

Azeotropic Column Distillate 46:

There is a reasonably good agreement between measured and simulated data for the azeotropic column distillate streams (tables 13 and 17). In the light phase, the concentrations 1-octene and n-octane compare particularly well. The simulation predicts considerably less acetonitrile in the light phase than was measured. In the heavy phase, the simulation predicts comparable water and acetonitrile concentrations, although the acetonitrile concentration is somewhat lower than that determined experimentally. The simulation also predicts between 1.5 to 2 times the amount of hydrocarbons (C₈ fraction) in the heavy phase than was measured.

Stripper Column Distillate 48:

The phase separation of the stripper column distillate stream is not approximated well by the simulation (tables 15 and 19). In the light phase, there is only good agreement for n-octane. The simulation predicted significantly higher 1-octene, and significantly lower acetonitrile concentrations than was measured. These differences are more marked for data point 4 than for data point 5. In the heavy phase, the simulation predicted close to double the hydrocarbon concentration (C₈ fraction) and significantly lower acetonitrile concentrations than was experimentally determined.

TABLE 12
Azeotropic Column Results for Data Point 4

| | Input | | Results | Plant Data |
|---|---|---|---|---|
| Component | Feed | Solvent | Bottoms | Bottoms |
| Toluene | 0.909 | 0.349 | 0.015 | 0.164 |
| 1-Octene | 51.569 | 2.334 | 0.289 | 0.203 |
| n-Octane | 8.498 | 0.172 | 0.037 | 0.049 |
| Ethyl Benzene | 0.108 | 0.027 | 0.796 | 0.457 |
| Butyl Acetate | 0.076 | 0.006 | 0.570 | 0.358 |
| 2-Hexanone | 5.886 | 0.048 | 40.055 | 41.429 |
| Hexanal | 0.511 | 0.049 | 4.017 | 2.628 |
| 1-Butanol | 0.076 | 0.040 | 0.015 | 0.053 |
| 1-Pentanol | 2.824 | 0.000 | 18.994 | 17.186 |
| Propanoic Acid | 0.999 | 0.000 | 6.723 | 4.960 |
| Isobutanoic Acid | 0.781 | 0.000 | 5.253 | 4.687 |
| Butanoic Acid | 0.059 | 0.000 | 0.522 | 0.379 |
| Water | 0.000 | 15.355 | 0.000 | n.a. |
| Acetonitrile | 0.000 | 79.855 | 0.000 | 0.000 |
| Flow Rate (kg/hr) | 0.800 | 1.939 | 0.1180 | 0.088 |
| Temperature (° C.) | 105.0 | 55.0 | 128.7 | 130.0 |

| | |
|---|---|
| Theoretical Stages | 18 |
| Feed Stage | 8 |

TABLE 13
Azeotropic Column Distillate for Data Point 4

| | Simu- | | | | |
|---|---|---|---|---|---|
| | lation | Heavy | | Light | |
| | Results | Phase | Heavy | Phase | Light |
| | for | Simu- | Phase | Simu- | Phase |
| | Total | lation | Plant | lation | Plant |
| Component | Distillates | Result | Data | Result | Data |
| Toluene | 0.533 | 0.370 | 0.369 | 1.021 | 1.018 |
| 1-Octene | 17.331 | 4.656 | 2.233 | 55.317 | 55.911 |
| n-Octane | 2.699 | 0.363 | 0.176 | 9.702 | 9.072 |
| 2-Hexanone | 0.014 | 0.017 | 0.036 | 0.007 | 0.060 |
| Hexanal | 0.010 | 0.011 | 0.037 | 0.008 | 0.040 |
| Water | 11.421 | 15.148 | 14.5 | 0.249 | 0.05 |
| Acetonitrile | 59.076 | 77.534 | 80.291 | 3.761 | 5.325 |
| Flow Rate | 2.6210 | | | | |
| (kg/hr) | | | | | |
| Temper- | 68.9 | 30.0 | | 30.0 | |
| ature (° C.) | | | | | |

TABLE 14
Stripper Column Results for Data Point 4

| | Input | Simulation Result | Plant Data |
|---|---|---|---|
| Component | Feed | Bottoms | Bottoms |
| Toluene | 0.941 | 0.980 | 0.990 |
| 1-Octene | 55.011 | 58.777 | 59.250 |
| n-Octane | 9.136 | 9.810 | 9.828 |
| 2-Hexanone | 0.023 | 0.025 | — |
| Hexanal | 0.019 | 0.020 | — |
| Water | 0.050 | 0.000 | n.a. |
| Acetonitrile | 6.053 | 0.000 | 0.000 |
| Flow Rate (kg/hr) | 0.7720 | 0.0900 | 0.6500 |
| Temperature (° C.) | 50.0 | 114.6 | 114.0 |

| | |
|---|---|
| Theoretical Stages | 8 |

TABLE 15
Stripper Column Distillate for Data Point 4

| | Simu- | | | | |
|---|---|---|---|---|---|
| | lation | Heavy | | Light | |
| | Results | Phase | Heavy | Phase | Light |
| | for | Simu- | Phase | Simu- | Phase |
| | Total | lation | Plant | lation | Plant |
| Component | Distillate | Result | Data | Result | Data |
| Toluene | 0.647 | 0.632 | 0.303 | 0.679 | 0.419 |
| 1-Octene | 26.473 | 14.561 | 7.448 | 51.151 | 46.930 |
| n-Octane | 4.032 | 1.577 | 0.884 | 9.119 | 9.281 |
| 2-Hexanone | 0.011 | 0.014 | 0.061 | 0.004 | 0.014 |
| Hexanal | 0.007 | 0.009 | 0.025 | 0.004 | 0.012 |
| Water | 0.429 | 0.614 | 1.02 | 0.046 | 0.03 |
| Acetonitrile | 51.920 | 74.453 | 84.030 | 5.237 | 9.349 |
| Flow Rate | 0.6820 | | | | |
| (kg/hr) | | | | | |
| Temper- | 73.2 | 33.0 | | 33.0 | |
| ature (° C.) | | | | | |

TABLE 16
Azeotropic Column Results for Data Point 5

| | Input | | Results | Plant Data |
|---|---|---|---|---|
| Component | Feed | Solvent | Bottoms | Bottoms |
| Toluene | 0.909 | 0.081 | 0.009 | 0.231 |
| 1-Octene | 51.569 | 2.632 | 19.009 | 19.011 |
| n-Octane | 8.498 | 0.196 | 4.388 | 4.487 |
| Ethyl Benzene | 0.108 | 0.000 | 0.514 | 0.487 |
| Butyl Acetate | 0.076 | 0.000 | 0.361 | 0.276 |
| 2-Hexanone | 5.886 | 0.039 | 28.374 | 26.301 |
| Hexanal | 0.511 | 0.000 | 2.437 | 1.951 |
| 1-Butanol | 0.076 | 0.215 | 0.233 | 0.089 |
| 1-Pentanol | 2.824 | 0.000 | 13.468 | 11.402 |
| Propanoic Acid | 0.999 | 0.000 | 4.766 | 3.530 |
| Isobutanoic Acid | 0.781 | 0.000 | 3.724 | 3.162 |
| Butanoic Acid | 0.059 | 0.000 | 0.283 | 0.231 |
| Water | 0.000 | 14.927 | 0.000 | n.a. |
| Acetonitrile | 0.000 | 79.728 | 0.000 | 0.0014 |
| Flow Rate (kg/hr) | 0.788 | 1.789 | 0.16525 | 0.1810 |
| Temperature (° C.) | 105.0 | 40.0 | 118.9 | 119.2 |

| | |
|---|---|
| Theoretical Stages | 19 |
| Feed Stage | 9 |

TABLE 17
Azeotropic Column Distillate for Data Point 5

| | Simulation | | | | |
|---|---|---|---|---|---|
| | Results for | Heavy Phase | | Light Phase | |
| | Total | Simulation | Heavy Phase | Simulation | Light Phase |
| Component | Distillate | Result | Plant Data | Result | Plant Data |
| Toluene | 0.417 | 0.401 | 0.381 | 1.076 | 1.043 |
| 1-Octene | 17.499 | 4.848 | 2.678 | 55.512 | 56.100 |
| n-Octane | 2.621 | 0.368 | 0.233 | 9.392 | 9.020 |
| 2-Hexanone | 0.008 | 0.009 | 0.039 | 0.004 | — |
| Hexanal | 0.000 | 0.000 | — | 0.000 | — |
| Water | 11.073 | 14.674 | 14.50 | 0.252 | n.a. |
| Acetonitrile | 59.141 | 77.560 | 80.277 | 3.799 | 5.553 |
| Flow Rate (kg/hr) | 2.4118 | | | | |
| Temperature (° C.) | 68.6 | 30.0 | | 30.0 | |

TABLE 18
Stripper Column Results for Data Point 5

| Component | Input | Simulation Result | Plant Data |
|---|---|---|---|
| | Feed | Bottoms | Bottoms |
| Toluene | 1.040 | 1.087 | 1.116 |
| 1-Octene | 56.136 | 59.628 | 60.250 |
| n-Octane | 9.001 | 9.595 | 9.370 |
| 2-Hexanone | 0.000 | 0.000 | — |
| Hexanal | 0.000 | 0.000 | — |
| Water | 0.050 | 0.000 | n.a. |
| Acetonitrile | 5.541 | 0.014 | 0.000 |
| Flow Rate (kg/hr) | 0.6950 | 0.6227 | 0.5420 |
| Temperature (° C.) | 50.0000 | 113.6739 | 113.6 |

| | |
|---|---|
| Theoretical Stages | 8 |

TABLE 19
Stripper Column Distillate for Data Point 5

| | Simulation | | | | |
|---|---|---|---|---|---|
| | Results for | Heavy Phase | | Light Phase | |
| | Total | Simulation | Heavy Phase | Simulation | Light Phase |
| Component | Distillate | Result | Plant Data | Result | Plant Data |
| Toluene | 0.638 | 0.623 | 0.300 | 0.672 | 0.408 |
| 1-Octene | 26.062 | 14.449 | 7.344 | 51.514 | 45.846 |
| n-Octane | 3.882 | 1.531 | 0.869 | 9.035 | 9.000 |
| 2-Hexanone | 0.000 | 0.000 | 0.133 | 0.000 | 0.043 |
| Hexanal | 0.000 | 0.000 | — | 0.000 | — |
| Water | 0.479 | 0.676 | 1.00 | 0.049 | n.a. |
| Acetonitrile | 53.141 | 75.039 | 82.892 | 5.148 | 6.441 |
| Flow Rate (kg/hr) | 0.0723 | | | | |
| Temperature (° C.) | 73.7 | 33.0 | 33.0 | 33.0 | 33.0 |

The octene recovery for data point 4 was in excess of the desired 98.5%. In both design runs analyzed here, the hexanal specification on the sweetened C₈ (stripper bottoms) stream was met. The acetonitrile specification was met in both bottoms streams for data point 4, and was met for the stripper bottoms in data point 5.

TABLE 20
GC Analysis of C₈ broadcut

| Retention | | | |
|---|---|---|---|
| Time | Mass % | GC-MS Identification | Simulation |
| 2.511 | 0.005068 | Hydrocarbon | Hydrocarbon |
| 2.977 | 0.043491 | Hydrocarbon | Hydrocarbon |
| 3.041 | 0.005582 | Hydrocarbon | Hydrocarbon |
| 3.083 | 0.012221 | Hydrocarbon | Hydrocarbon |
| 3.194 | 0.006287 | Hydrocarbon | Hydrocarbon |
| 3.24 | 0.147933 | Hydrocarbon | Hydrocarbon |
| 3.303 | 1.23937 | Hydrocarbon | Hydrocarbon |
| 3.351 | 0.639047 | Hydrocarbon | Hydrocarbon |
| 3.407 | 2.074029 | Hydrocarbon | Hydrocarbon |
| 3.451 | 0.356071 | Hydrocarbon | Hydrocarbon |
| 3.528 | 0.097393 | Hydrocarbon | Hydrocarbon |
| 3.584 | 0.75468 | Hydrocarbon | Hydrocarbon |
| 3.665 | 8.448708 | n-octane | N-OCTANE |
| 3.717 | 3.393533 | Hydrocarbon | Hydrocarbon |
| 3.868 | 3.130383 | Hydrocarbon | Hydrocarbon |
| 3.924 | 0.796352 | Hydrocarbon | Hydrocarbon |
| 3.984 | 0.32873 | Hydrocarbon | Hydrocarbon |
| 4.036 | 0.493326 | Hydrocarbon | Hydrocarbon |
| 4.109 | 0.182206 | Hydrocarbon | Hydrocarbon |
| 4.259 | 51.26867 | 1-octene | 1-OCTENE |
| 4.31 | 0.075931 | Hydrocarbon | Hydrocarbon |
| 4.422 | 0.36444 | Hydrocarbon | Hydrocarbon |
| 4.466 | 1.053717 | Hydrocarbon | Hydrocarbon |
| 4.51 | 0.224041 | Hydrocarbon | Hydrocarbon |
| 4.618 | 0.956411 | Hydrocarbon | Hydrocarbon |
| 4.677 | 1.801322 | Hydrocarbon | Hydrocarbon |
| 4.765 | 0.171383 | Hydrocarbon | Hydrocarbon |
| 4.846 | 0.379639 | Hydrocarbon | Hydrocarbon |
| 4.959 | 0.167541 | Hydrocarbon | Hydrocarbon |
| 5.052 | 0.250624 | Hydrocarbon | Hydrocarbon |
| 5.151 | 0.371768 | Hydrocarbon | Hydrocarbon |
| 5.204 | 0.668217 | Hydrocarbon | Hydrocarbon |
| 5.338 | 0.449502 | Hydrocarbon | Hydrocarbon |
| 5.382 | 0.228238 | Hydrocarbon | Hydrocarbon |
| 5.473 | 0.087151 | Hydrocarbon | Hydrocarbon |
| 5.574 | 0.336488 | Hydrocarbon | Hydrocarbon |
| 5.635 | 1.136021 | Hydrocarbon | Hydrocarbon |
| 5.852 | 1.134322 | Hydrocarbon | Hydrocarbon |
| 5.942 | 0.972871 | Hydrocarbon | Hydrocarbon |
| 6.077 | 0.124791 | Hydrocarbon | Hydrocarbon |
| 6.24 | 0.211384 | Hydrocarbon | Hydrocarbon |
| 6.476 | 0.058137 | Hydrocarbon | Hydrocarbon |
| 6.565 | 0.056595 | Hydrocarbon | Hydrocarbon |
| 6.733 | 0.07084 | Hydrocarbon | Hydrocarbon |
| 6.935 | 0.031847 | Hydrocarbon | Hydrocarbon |
| 7.105 | 0.031424 | Hydrocarbon | Hydrocarbon |
| 7.265 | 0.009995 | Hydrocarbon | Hydrocarbon |
| 7.412 | 0.03435 | Hydrocarbon | Hydrocarbon |
| 7.749 | 0.013635 | Hydrocarbon | Hydrocarbon |
| 7.823 | 0.06842 | Cyclic Hydrocarbon | 1-METHYL-1- |
| | | | ETHYCYCLO- |
| | | | PENTANE |
| 8.19 | 0.064099 | 2-methylpentanal | 1-METHYLPENTANAL |
| 8.229 | 0.046491 | MIBK | MIBK |
| 8.639 | 0.082199 | 3-methylpentanal | 1-METHYLPENTANAL |
| 8.854 | 0.904193 | Tolueen | TOLUENE |
| 9.071 | 0.38793 | 3-hexanone | 3-HEXANONE |
| 9.557 | 0.075291 | butylacetate | N-BUTYL-ACETATE |
| 9.669 | 0.025217 | C₇ketone | 5-METHYL-2- |
| | | | HEXANONE |
| 9.799 | 5.851833 | 2-hexanone | 2-HEXANONE |
| 9.848 | 0.508014 | hexanal | 1-HEXANAL |
| 10.023 | 0.025718 | C₇ketone | 5-METHYL-2- |
| | | | HEXANONE |
| 10.383 | 0.06678 | C₇ketone | 5-METHYL-2- |
| | | | HEXANONE |
| 10.489 | 0.009959 | C₇ketone | 5-METHYL-2- |
| | | | HEXANONE |
| 10.709 | 0.056065 | C₇ketone | 5-METHYL-2- |
| | | | HEXANONE |
| 10.972 | 0.107845 | ethylbenzene | ETHYLBENZENE |
| 11.072 | 0.076346 | 1-butanol | N-BUTANOL |
| 11.169 | 0.027141 | 4-methyl-2-pentanol | 4-METHYL-2- |
| | | | PENTANOL |
| 11.525 | 0.013734 | cyclopentanone | CYCLOPENTANONE |
| 12.189 | 0.025914 | cyclopentanone | CYCLOPENTANONE |
| 12.34 | 0.166595 | 3-hexanol | 2-HEXANOL |
| 12.432 | 0.228465 | cyclopentanone | CYCLOPENTANONE |
| 12.48 | 0.60214 | 2-methyl-1-butanol | 2-METHYL-1- |
| | | | BUTANOL |
| 12.54 | 0.021664 | cyclopentanone | CYCLOPENTANONE |
| 12.678 | 0.225832 | cyclopentanone | CYCLOPENTANONE |
| 12.95 | 0.29267 | 2-hexanol | 2-HEXANOL |
| 13.242 | 0.032278 | pentyl propionate | N-BUTYL-N- |
| | | | BUTYRATE |
| 13.312 | 2.808017 | 1-pentanol | 1-PENTANOL |
| 13.707 | 0.017209 | branched C₆ alcohol | 2-HEXANOL |
| 14.259 | 0.199871 | 2-methyl-1-pentanol | 2-METHYL-1- |
| | | | PENTANOL |
| 14.401 | 0.083215 | 2-ethyl-1-butanol | 2-ETHYL-1- |
| | | | BUTANOL |
| 14.518 | 0.098982 | 4-methyl-1-pentanol | 2-METHYL-1- |
| | | | PENTANOL |
| 14.764 | 0.035969 | 3-methyl-1-pentanol | 2-METHYL-1- |
| | | | PENTANOL |
| 15.084 | 0.021057 | C₆ alcohol | 2-HEXANOL |
| 18.34 | 0.003604 | propanoic acid | PROPIONIC-ACID |
| 18.765 | 0.7764 | isobutanoic acid | ISOBUTYRIC-ACID |
| 19.641 | 0.058977 | butanoic acid | N-BUTYRIC-ACID |
| 22.512 | 0.018163 | phenol | N-BUTYRIC-ACID |

TABLE 21
Aspen ™ Simulation Stream Results for Data Point 4
Azeotropic Column

| | Mass Fractions | | | |
|---|---|---|---|---|
| Component | Feed | Reflux | Distillate | Bottoms |
| 2-METHYL-2-PENTENE | 8.60E−07 | 5.14E−08 | 3.01E−07 | 6.57E−16 |
| 1-HEPTENE | 0.00221789 | 0.0001327 | 0.0007751 | 2.47E−09 |
| N-HEPTANE | 0.00035861 | 2.15E−05 | 0.0001253 | 2.72E−10 |
| 2,3-DIMETHYL-1-HEXENE | 0.01083749 | 0.0006483 | 0.0037875 | 1.27-E−06 |
| TOLUENE | 0.00902388 | 0.003486 | 0.0053265 | 0.00015 |
| 2-METHYL-1-HEPTENE | 0.089273 | 0.0053407 | 0.0311983 | 2.74E−05 |
| 3-METHYLHEPTANE | 0.053963 | 0.0032283 | 0.0188582 | 2.24E−05 |
| 2-METHYL-1-HEPTENE | 2.60E−02 | 0.0015577 | 0.0090971 | 5.84E−05 |
| TRANS-1,4-DIMETHYLCYCLOHEXANE | 6.84E−03 | 0.0004093 | 0.0023908 | 9.33E−06 |
| 2-ETHYL-1-HEXENE | 3.82E−03 | 0.0002287 | 0.0013354 | 1.11E−05 |
| 1-OCTENE | 0.51166335 | 0.0233422 | 0.1733116 | 0.002893 |
| TRANS-4-OCTENE | 0.00047041 | 2.81E−05 | 0.0001643 | 2.98E−06 |
| 1-METHYL-1-ETHYLCYCLOPENTANE | 0.01148075 | 0.0006868 | 0.0040114 | 2.17E−05 |
| TRANS-2-OCTENE | 0.01228398 | 0.0007349 | 0.0042839 | 0.000204 |
| CIS-2-OCTENE | 0.00964383 | 0.0005769 | 0.0033609 | 0.00021 |
| N-OCTANE | 0.08431844 | 0.0017224 | 0.0269939 | 0.000368 |
| 2,2-DIMETHYLHEPTANE | 0.01206124 | 0.0007216 | 0.0041951 | 0.000447 |
| 2,6-DIMETHYLHEPTANE | 0.00571888 | 0.0003421 | 0.0019702 | 0.000632 |
| ETHYLBENZENE | 0.00107629 | 0.0002664 | 0.0001673 | 0.007958 |
| 1ECHEXE | 0.00030443 | 1.82E−05 | 7.53E−05 | 0.000691 |
| P-XYLENE | 0.00288265 | 0.0001725 | 0.0001052 | 0.020041 |
| 4-METHYLOCTANE | 0.00037237 | 2.23E−05 | 0.0001069 | 0.000517 |
| 3-METHYLOCTANE | 0.00016769 | 1.00E−05 | 4.12E−05 | 0.000387 |
| 2M1OCTE | 0.00083676 | 5.01E−05 | 9.38E−05 | 0.004412 |
| 1-NONENE | 0.0017019 | 0.0001018 | 0.0001069 | 0.010837 |
| 1-DECENE | 2.58E−06 | 1.54E−07 | 8.45E−08 | 1.81E−05 |
| 1-METHYL-1-ETHYLCYCLOPENTANE | 0.00068283 | 0.002627 | 0.0021518 | 7.97E−07 |
| ETHYLCYCLOHEXANE | 0 | 0 | 0 | 0 |
| 2M1PNTAN | 0.00146007 | 0 | 2.98E−05 | 0.009236 |
| METHYL-ISOBUTYL-KETONE | 0.00046398 | 0 | 6.79E−05 | 0.001637 |
| ETHYL-BUTYRATE | 0 | 0 | 0 | 0 |
| N-PROPYL-PROPIONATE | 0 | 0 | 0 | 0 |
| 3-HEXANONE | 0.00387155 | 0 | 5.41E−05 | 0.025047 |
| DIISOPROPYL-KETONE | 0 | 0 | 0 | 0 |
| N-BUTYL-ACETATE | 0.0007514 | 6.10E−05 | 1.79E−05 | 0.005699 |
| 2-HEXANONE | 0.0584015 | 0.0004751 | 0.000144 | 0.40055 |
| 1-HEXANAL | 0.00506999 | 0.0004867 | 9.91E−05 | 0.04017 |
| 5-METHYL-2-HEXANONE | 0.00183372 | 0 | 1.55E−09 | 0.012432 |
| N-BUTANOL | 0.00076193 | 0.0004024 | 0.0005234 | 0.000153 |
| 4-METHYL-2-PENTANOL | 0.00027086 | 0 | 1.11E−06 | 0.001812 |
| CYCLOPENTANONE | 0.00514579 | 0 | 2.73E−06 | 0.034826 |
| 2-METHYL-1-BUTANOL | 6.01E−03 | 0 | 5.73E−05 | 0.039468 |
| 3-METHYL-1-BUTANOL | 0 | 0 | 0 | 0 |
| 2-HEXANOL | 0.00496537 | 0 | 1.44E−07 | 0.03366 |
| N-BUTYL-N-BUTYRATE | 3.22E−04 | 0 | 9.58E−12 | 0.002184 |
| 1-PENTANOL | 2.80E−02 | 0 | 2.51E−06 | 0.189939 |
| 2-ETHYL-1-BUTANOL | 8.30E−04 | 0 | 3.32E−09 | 0.00563 |
| 2-HEPTANONE | 0 | 0 | 0 | 0 |
| 2-METHYL-1-PENTANOL | 3.34E−03 | 0 | 5.34E−09 | 0.022654 |
| PROPIONIC-ACID | 9.92E−03 | 0 | 2.31E−09 | 0.067229 |
| ISOBUTYRIC-ACID | 0.0077485 | 0 | 4.40E−12 | 0.052532 |
| N-BUTYRIC-ACID | 0.00076986 | 0 | 1.03E−14 | 0.005219 |
| WATER | 0.001996 | 0.1535509 | 0.1142052 | 6.70E−16 |
| ACETONITRILE | 0 | 0.7985474 | 0.5907605 | 7.26E−10 |
| Total Flow (mol/sec) | 0.00205152 | 0.0153019 | 0.0170067 | 3.47E−04 |
| Total Flow (kg/hr) | 0.8 | 1.939 | 2.621 | 0.17999 |
| Total Flow (m³/hr) | 0.00229331 | 0.0026735 | 1.9783793 | 0.000164 |
| Temperature (° C.) | 105 | 55 | 68.937559 | 1.28E+02 |
| Pressure (bar) | 0.89 | 0.87 | 0.86 | 9.10E−01 |
| Vapor Fraction | 0.00434105 | 0 | 1 | 0.00E+00 |
| Enthalpy (Mmkcal/hr) | −0.2901608 | −0.750563 | −0.432572 | −0.096777 |

TABLE 22
Aspen ™ Simulation Stream Results for Data Point 4
Stripper Column

| | Mass Fractions | | |
|---|---|---|---|
| Component | Feed | Distillate | Bottoms |
| 2-METHYL-2-PENTENE | 9.73E−07 | 6.69E−06 | 2.18E−07 |
| 1-HEPTENE | 0.00250822 | 0.0037091 | 0.0023498 |
| N-HEPTANE | 0.00040555 | 0.0005309 | 0.000389 |
| 2,3-DIMETHYL-1-HEXENE | 0.01225614 | 0.0088657 | 0.0127036 |
| TOLUENE | 0.00941027 | 0.0064701 | 0.0097983 |
| 2-METHYL-1-HEPTENE | 0.10095904 | 0.0661221 | 0.1055563 |
| 3-METHYLHEPTANE | 0.06102688 | 0.0349999 | 0.0644615 |
| 2-METHYL-1-HEPTENE | 0.02944606 | 0.0157073 | 0.0312591 |
| TRANS-1,4-DIMETHYLCYCLOHEXANE | 0.00773765 | 0.0046325 | 0.0081474 |
| 2-ETHYL-1-HEXENE | 0.004323 | 0.0022179 | 0.0046008 |
| 1-OCTENE | 0.55011376 | 0.264728 | 0.5877747 |
| TRANS-4-OCTENE | 0.00053198 | 0.0002565 | 0.0005683 |
| 1-METHYL-1-ETHYLCYCLOPENTANE | 0.01298361 | 0.0068376 | 0.0137947 |
| TRANS-2-OCTENE | 0.01389198 | 0.0060991 | 0.0149204 |
| CIS-2-OCTENE | 0.01090623 | 0.0036747 | 0.0117286 |
| N-OCTANE | 0.09136175 | 0.0403216 | 0.0980973 |
| 2,2-DIMETHYLHEPTANE | 0.01364009 | 0.0048454 | 0.0148007 |
| 2,6-DIMETHYLHEPTANE | 0.00646749 | 0.0020079 | 0.007056 |
| ETHYLBENZENE | 0.00091642 | 0.0002402 | 0.0010057 |
| 1ECHEXE | 3.44E−04 | 1.04E−04 | 3.76E−04 |
| P-XYLENE | 0.00326 | 0.0008539 | 0.0035775 |
| 4-METHYLOCTANE | 0.00042111 | 0.000102 | 0.0004632 |
| 3-METHYLOCTANE | 0.00018964 | 4.305E−05 | 0.000209 |
| 2M1OCTE | 0.00094629 | 0.0002065 | 0.0010439 |
| 1-NONENE | 0.00192468 | 0.0003628 | 0.0021308 |
| 1-DECENE | 2.92E−06 | 2.22E−07 | 3.27E−06 |
| 1-METHYL-1-ETHYLCYCLOPENTANE | 0 | 0 | 0 |
| ETHYLCYCLOHEXANE | 0 | 0 | 0 |
| 2M1PNTAN | 0.00214482 | 0.0011852 | 0.0022715 |
| METHYL-ISOBUTYL-KETONE | 0 | 0 | 0 |
| ETHYL-BUTYRATE | 0 | 0 | 0 |
| N-PROPYL-PROPIONATE | 0 | 0 | 0 |
| 3-HEXANONE | 0.00043201 | 0.0002066 | 0.0004618 |
| DIISOPROPYL-KETONE | 0 | 0 | 0 |
| N-BUTYL-ACETATE | 0 | 0 | 0 |
| 2-HEXANONE | 0.00023 | 0.0001073 | 0.0002462 |
| 1-HEXANAL | 0.0001886 | 0.0000699 | 0.0002043 |
| 5-METHYL-2-HEXANONE | 0 | 0 | 0 |
| N-BUTANOL | 0 | 0 | 0 |
| 4-METHYL-2-PENTANOL | 0 | 0 | 0 |
| CYCLOPENTANONE | 0 | 0 | 0 |
| 2-METHYL-1-BUTANOL | 0 | 0 | 0 |
| 3-METHYL-1-BUTANOL | 0 | 0 | 0 |
| 2-HEXANOL | 0 | 0 | 0 |
| N-BUTYL-N-BUTYRATE | 0 | 0 | 0 |
| 1-PENTANOL | 0 | 0 | 0 |
| 2-ETHYL-1-BUTANOL | 0 | 0 | 0 |
| 2-HEPTANONE | 0 | 0 | 0 |
| 2-METHYL-1-PENTANOL | 0 | 0 | 0 |
| PROPIONIC-ACID | 0 | 0 | 0 |
| ISOBUTYRIC-ACID | 0 | 0 | 0 |
| N-BUTYRIC-ACID | 0 | 0 | 0 |
| WATER | 0.00049995 | 0.0042885 | 8.00E−12 |
| ACETONITRILE | 0.06052846 | 0.5191961 | 4.78E−07 |
| Temperature (° C.) | 50 | 74.691222 | 114.62583 |
| Pressure (bar) | 0.87 | 0.86 | 0.865 |
| Vapor Fraction | 0 | 1 | 0 |
| Mole Flow (mol/sec) | 0.00211167 | 0.0004283 | 0.0016834 |
| Mass Flow (kg/hr) | 0.772 | 0.09 | 0.682 |
| Volume Flow (m³/hr) | 0.00106096 | 0.0502664 | 0.0010583 |
| Enthalpy (Mmkcal/hr) | −0.2107546 | 0.0105329 | −0.183224 |

TABLE 23
Aspen ™ Simulation Results for Data Point 5
Azeotropic Column

| | Mass Fractions | | | |
|---|---|---|---|---|
| Component | Feed | Reflux | Distillate | Bottoms |
| 2-METHYL-2-PENTENE | 8.67E−07 | 6.46E−08 | 3.31E−07 | 2.57E−14 |
| 1-HEPTENE | 0.00223533 | 0.00016649 | 0.0008539 | 1.25E−07 |
| N-HEPTANE | 0.00036143 | 2.69E−05 | 0.0001381 | 2.64E−08 |
| 2,3-DIMETHYL-1-HEXENE | 0.01092271 | 0.00081356 | 0.0041658 | 9.44E−05 |
| TOLUENE | 0.00909484 | 0.00377664 | 0.0056981 | 0.0010945 |
| 2-METHYL-1-HEPTENE | 0.08997503 | 0.00670167 | 0.0342298 | 0.0020324 |
| 3-METHYLHEPTANE | 0.05438736 | 0.00405097 | 0.0205735 | 0.0029431 |
| 2-METHYL-1-HEPTENE | 0.02624242 | 0.00195463 | 0.0097319 | 0.0042667 |
| TRANS-1,4-DIMETHYLCYCLOHEXANE | 0.00689581 | 0.00051362 | 0.0025952 | 0.000567 |
| 2-ETHYL-1-HEXENE | 0.00385268 | 0.00028696 | 0.0014152 | 0.0008242 |
| 1-OCTENE | 0.51568698 | 0.02631877 | 0.1749905 | 0.1900901 |
| TRANS-4-OCTENE | 0.0004741 | 3.53E−05 | 0.0001675 | 0.000199 |
| 1-METHYL-1-ETHYLCYCLOPENTANE | 0.01157104 | 0.00086185 | 0.004289 | 0.0019114 |
| TRANS-2-OCTENE | 0.0123806 | 0.0009222 | 0.0039298 | 0.0116668 |
| CIS-2-OCTENE | 0.00971967 | 0.00072395 | 0.0029426 | 0.0112401 |
| N-OCTANE | 0.0849815 | 0.00195667 | 0.0262112 | 0.0438792 |
| 2,2-DIMETHYLHEPTANE | 0.01215609 | 0.00090543 | 0.0027237 | 0.0280179 |
| 2,6-DIMETHYLHEPTANE | 0.00576385 | 0.00042931 | 0.0008814 | 0.019269 |
| ETHYLBENZENE | 0.00108476 | 0 | 1.97E−06 | 0.005144 |
| 1ECHEXE | 0.00030682 | 2.29E−05 | 2.35E−05 | 0.0013678 |
| P-XYLENE | 0.00290532 | 0.00021639 | 0.0001163 | 0.0144989 |
| 4-METHYLOCTANE | 0.0003753 | 2.80E−05 | 2.96E−05 | 0.0016607 |
| 3-METHYLOCTANE | 0.0001690 | 0.000013 | 0.000012 | 0.0007656 |
| 2M1OCTE | 0.00084334 | 0.000063 | 0.000051 | 0.0039606 |
| 1-NONENE | 0.001715 | 0.000128 | 0.000093 | 0.0081994 |
| 1-DECENE | 2.60E−06 | 1.94E−07 | 1.00E−07 | 1.30E−05 |
| 1-METHYL-1-ETHYLCYCLOPENTANE | 0.0006882 | 0 | 0.0002199 | 7.17E−05 |
| ETHYLCYCLOHEXANE | 0 | 0 | 0 | 0 |
| 2M1PNTAN | 0.00146854 | 0 | 2.34E−06 | 0.0069686 |
| METHYL-ISOBUTYL-KETONE | 0.00050292 | 0 | 1.28E−05 | 0.002212 |
| ETHYL-BUTYRATE | 0 | 0 | 0 | 0 |
| N-PROPYL-PROPIONATE | 0 | 0 | 0 | 0 |
| 3-HEXANONE | 0.003902 | 0 | 3.31E−06 | 0.0185586 |
| DIISOPROPYL-KETONE | 0 | 0 | 0 | 0 |
| N-BUTYL-ACETATE | 0.0007573 | 0 | 3.94E−08 | 0.0036107 |
| 2-HEXANONE | 0.0588608 | 0.00038913 | 7.89E−05 | 0.283741 |
| 1-HEXANAL | 0.0051099 | 0 | 3.34E−08 | 0.0243657 |
| 5-METHYL-2-HEXANONE | 2.26E−03 | 0 | 1.62E−10 | 0.0107872 |
| N-BUTANOL | 0.0007627 | 0.00214924 | 0.0016837 | 0.0023313 |
| 4-METHYL-2-PENTANOL | 0 | 0 | 0 | 0 |
| CYCLOPENTANONE | 0 | 0 | 0 | 0 |
| 2-METHYL-1-BUTANOL | 0 | 0 | 0 | 0 |
| 3-METHYL-1-BUTANOL | 0.00011768 | 0 | 1.38E−08 | 0.000561 |
| 2-HEXANOL | 0.00294382 | 0 | 4.25E−09 | 0.0140377 |
| N-BUTYL-N-BUTYRATE | 0 | 0 | 0 | 0 |
| 1-PENTANOL | 0.02824449 | 0 | 1.15E−07 | 0.1346831 |
| 2-ETHYL-1-BUTANOL | 0.00837019 | 0 | 1.42E−09 | 0.0399135 |
| 2-HEPTANONE | 0.00013981 | 0 | 1.18E−12 | 0.0006667 |
| 2-METHYL-1-PENTANOL | 0.00336781 | 0 | 2.11E−10 | 0.0160595 |
| PROPIONIC-ACID | 0.00999418 | 0 | 8.76E−11 | 0.0476576 |
| ISOBUTYRIC-ACID | 0.00780943 | 0 | 5.98E−14 | 0.0372396 |
| N-BUTYRIC-ACID | 0.00059322 | 0 | 6.99E−17 | 0.0028288 |
| WATER | 0 | 0.14926978 | 0.1107261 | 3.88E−18 |
| ACETONITRILE | 0 | 0.79727623 | 0.5914076 | 2.58E−11 |
| Total Flow (mol/sec) | 0.00199525 | 0.01401295 | 0.0155527 | 0.0004555 |
| Total Flow (kg/hr) | 0.788 | 1.789 | 2.41175 | 0.16525 |
| Total Flow (m³/hr) | 0.00118443 | 0.00241014 | 1.8289356 | 0.0002364 |
| Temperature (° C.) | 105 | 40 | 68.62402 | 118.90637 |
| Pressure (bar) | 0.9 | 0.85 | 0.85 | 0.9 |
| Vapor Fraction | 0 | 0 | 1 | 0 |
| Liquid Fraction | 1 | 1 | 0 | 1 |
| Solid Fraction | 0 | 0 | 0 | 0 |
| Enthalpy (kJ/kmol) | −162557.29 | −56919.984 | −27806.01 | −277903 |
| Enthalpy (kJ/kg) | −1481.7728 | −1605.0428 | −645.527 | −2757.804 |
| Enthalpy (kJ/sec) | −0.3243436 | −0.7976171 | −0.432458 | −0.126591 |
| Entropy (kJ/kmol-K) | −653.63383 | −159.11155 | −103.4981 | −573.8553 |
| Entropy (kJ/kg-K) | −5.9581261 | −4.4866642 | −2.402748 | −5.694722 |
| Density (kmol/m³) | 6.06442123 | 20.9309461 | 0.0306133 | 6.9355028 |
| Density (kg/m³) | 665.294893 | 742.278709 | 1.3186631 | 698.88841 |
| Average MW | 109.704598 | 35.4632183 | 43.074901 | 100.76968 |

TABLE 24
Aspen ™ Simulation Results for Data Point 5
Stripper Column

| | Mass Fractions | | |
|---|---|---|---|
| Component | Feed | Distillate | Bottoms |
| 2-METHYL-2-PENTENE | 8.29E−07 | 2.89E−06 | 5.90E−07 |
| 1-HEPTENE | 0.00222718 | 0.00270089 | 0.0021722 |
| N-HEPTANE | 0.00043015 | 0.00049116 | 0.0004231 |
| 2,3-DIMETHYL-1-HEXENE | 0.01235251 | 0.00842136 | 0.0128089 |
| TOLUENE | 0.01040091 | 0.00638444 | 0.0108673 |
| 2-METHYL-1-HEPTENE | 0.10169535 | 0.06334433 | 0.1061482 |
| 3-METHYLHEPTANE | 0.06841953 | 0.03804184 | 0.0719466 |
| 2-METHYL-1-HEPTENE | 0.02946664 | 0.0151488 | 0.0311291 |
| TRANS-1,4-DIMETHYLCYCLOHEXANE | 0.00779627 | 0.00449613 | 0.0081794 |
| 2-ETHYL-1-HEXENE | 0.00431516 | 0.00213582 | 0.0045682 |
| 1-OCTENE | 0.56136107 | 0.26062454 | 0.5962788 |
| TRANS-4-OCTENE | 0.00052437 | 0.00024464 | 0.0005569 |
| 1-METHYL-1-ETHYLCYCLOPENTANE | 0.01397132 | 0.00715451 | 0.0147628 |
| TRANS-2-OCTENE | 0.01314158 | 0.0056031 | 0.0140169 |
| CIS-2-OCTENE | 0.0100963 | 0.00420782 | 0.0107800 |
| N-OCTANE | 0.09000748 | 0.0388240 | 0.0959503 |
| 2,2-DIMETHYLHEPTANE | 0.01263572 | 0.0044202 | 0.0135896 |
| 2,6-DIMETHYLHEPTANE | 0.00420843 | 0.0012872 | 0.0045476 |
| ETHYLBENZENE | 0 | 0 | 0 |
| 1ECHEXE | 7.20E−05 | 2.13E−05 | 7.79E−05 |
| P-XYLENE | 0.00025539 | 6.37E−05 | 0.0002777 |
| 4-METHYLOCTANE | 0.00010388 | 2.48E−05 | 0.0001131 |
| 3-METHYLOCTANE | 4.22E−05 | 9.46E−06 | 4.60E−05 |
| 2M1OCTE | 0.00016907 | 3.63E−05 | 0.0001845 |
| 1-NONENE | 0.00031472 | 5.82E−05 | 0.0003445 |
| 1-DECENE | 3.56E−07 | 2.66E−08 | 3.94E−07 |
| 1-METHYL-1-ETHYLCYCLOPENTANE | 0 | 0 | 0 |
| ETHYLCYCLOHEXANE | 0 | 0 | 0 |
| 2M1PNTAN | 0 | 0 | 0 |
| METHYL-ISOBUTYL-KETONE | 7.41E−05 | 4.46E−05 | 7.75E−05 |
| ETHYL-BUTYRATE | 0 | 0 | 0 |
| N-PROPYL-PROPIONATE | 0 | 0 | 0 |
| 3-HEXANONE | 1.07E−05 | 4.37E−06 | 1.15E−05 |
| DIISOPROPYL-KETONE | 0 | 0 | 0 |
| N-BUTYL-ACETATE | 0 | 0 | 0 |
| 2-HEXANONE | 0 | 0 | 0 |
| 1-HEXANAL | 0 | 0 | 0 |
| 5-METHYL-2-HEXANONE | 0 | 0 | 0 |
| N-BUTANOL | 0 | 0 | 0 |
| 4-METHYL-2-PENTANOL | 0 | 0 | 0 |
| CYCLOPENTANONE | 0 | 0 | 0 |
| 2-METHYL-1-BUTANOL | 0 | 0 | 0 |
| 3-METHYL-1-BUTANOL | 0 | 0 | 0 |
| 2-HEXANOL | 0 | 0 | 0 |
| N-BUTYL-N-BUTYRATE | 0 | 0 | 0 |
| 1-PENTANOL | 0 | 0 | 0 |
| 2-ETHYL-1-BUTANOL | 0 | 0 | 0 |
| 2-HEPTANONE | 0 | 0 | 0 |
| 2-METHYL-1-PENTANOL | 0 | 0 | 0 |
| PROPIONIC-ACID | 0 | 0 | 0 |
| ISOBUTYRIC-ACID | 0 | 0 | 0 |
| N-BUTYRIC-ACID | 0 | 0 | 0 |
| WATER | 0.00049881 | 0.00479492 | 6.58E−10 |
| ACETONITRILE | 0.05540794 | 0.5314085 | 0.0001408 |
| Total Flow (mol/sec) | 0.00188615 | 0.00034827 | 0.0015379 |
| Total Flow (kg/hr) | 0.695 | 0.07229999 | 0.6227 |
| Total Flow (m³/hr) | 0.00095829 | 0.04122056 | 0.0009659 |
| Temperature (° C.) | 50 | 73.4326375 | 113.6739 |
| Pressure (bar) | 0.85 | 0.85 | 0.85 |
| Vapor Fraction | 0 | 1 | 0 |
| Liquid Fraction | 1 | 0 | 1 |
| Solid Fraction | 0 | 0 | 0 |
| Enthalpy (kJ/kmol) | −118067.6 | 29656.1711 | −126537.8 |
| Enthalpy (kJ/kg) | −1153.522 | 514.289212 | −1125.032 |
| Enthalpy (kJ/sec) | −0.2226938 | 0.01032864 | −0.194599 |
| Entropy (kJ/kmol-K) | −638.39586 | −176.88227 | −672.5969 |
| Entropy (kJ/kg-K) | −6.2371355 | −3.0674441 | −5.979979 |
| Density (kmol/m³) | 7.08563747 | 0.03041701 | 5.7319263 |
| Density (kg/m³) | 725.243443 | 1.75397859 | 644.69719 |
| Average MW | 102.354015 | 57.664385 | 112.47479 |

### EXAMPLE 2

### Acid and Other Ogygenate Removal Using Azeotropic Distillation with Ethanol

An azeotropic distillation process to remove acids and oxygenates from C₈ broadcut using ethanol as the solvent was carried out in glass columns. It was aimed to firstly prove the process concept, and secondly to collect at least two sets of data point samples for the stripper and azeotropic columns, under stable operating conditions. The process was piloted without closing the solvent loop.

From the pilot plant experimental work it appears that the required 1-octene recovery of >98.5%, 1-hexanal specification of <100 ppm in the final product and ethanol concentrations of below 50 ppm in both column bottoms could be reached.

Stable operation of the phase separator and azeotropic column was possible between solvent water concentrations of 6.26 wt % and 9.77 wt %, operating at 28° C. At water concentration lower than 6.26 wt %, phase separation was lost, and at water concentrations higher than 9.77 wt %, there was phase separation in the azeotropic column below the feed point.

Phase separation is lost at 39° C., at a solvent water concentration at 9.3 wt %.

Aspen™ simulations have been able to approximate the results obtained on the pilot plant. The predicted product stream composition results match the experimental data well.

The same equipment and pilot plant configuration was used for the ethanol run as for Example 1. The phase separator was however operated at 28° C. to ensure stable phase separation.

As for Example 1, during the experiments five sets of data point samples were taken. All samples were analyzed, all flows and temperatures were plotted and mass balances were calculated where possible. All of this information was evaluated before a decision was taken whether the plant was stable for a long enough period when the samples were drawn, to warrant further processing of the data.

The criteria for stable operation are:

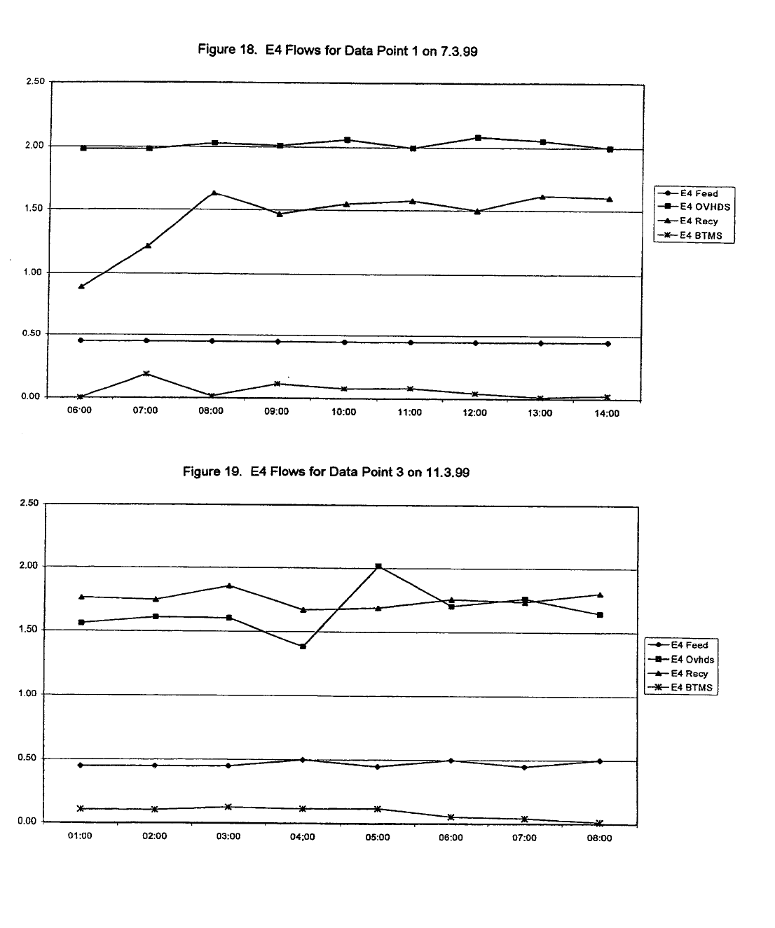

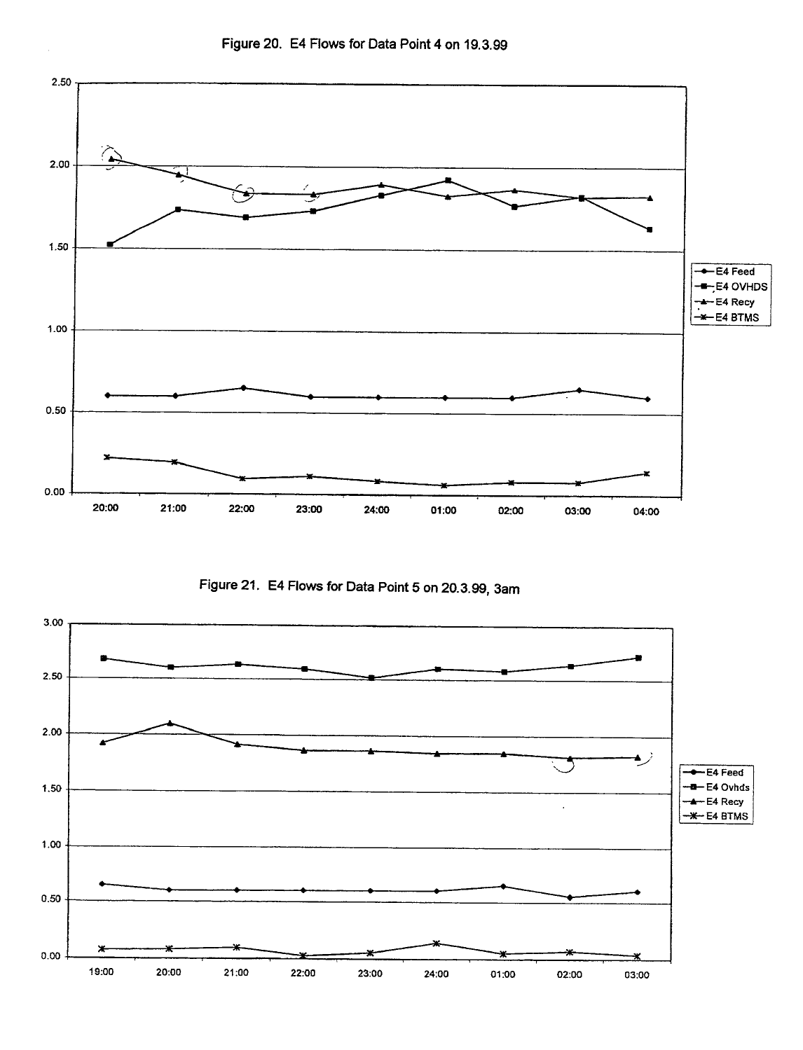

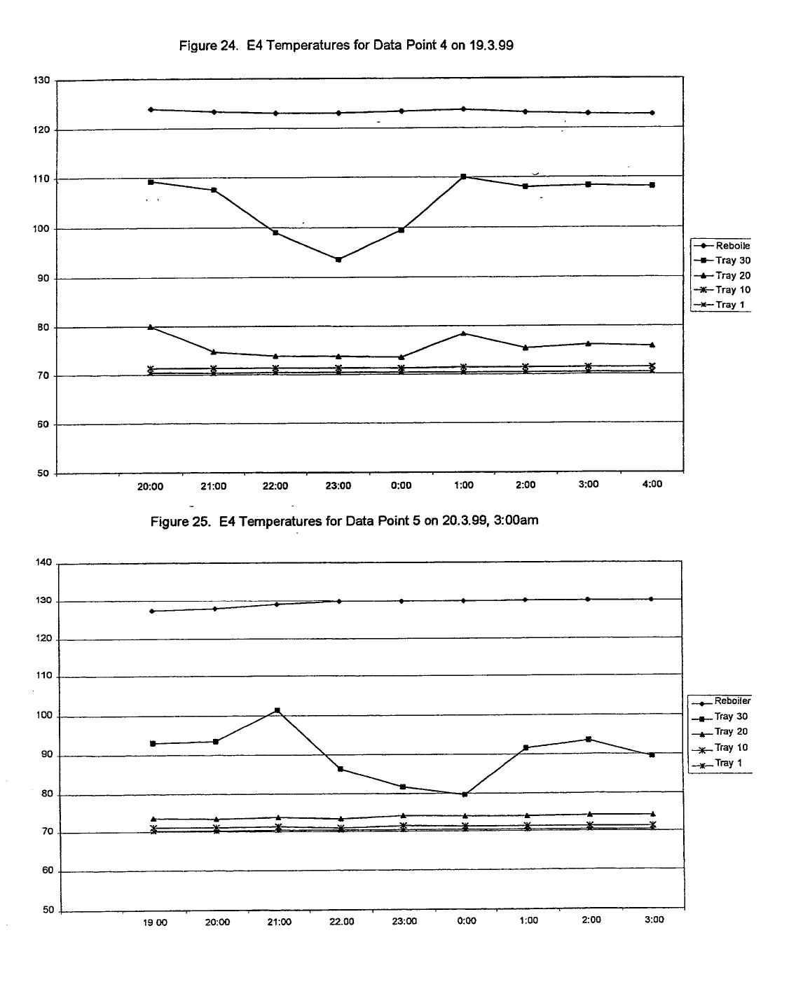

Constant feed and product flows as shown graphically in FIGS. 18 to 25.

The azeotropic column could be assessed in terms of constant flows, as the feed to this column was operated on flow control. The stripper column feed was operated on level control, to maintain constant level in the recycle stream buffer containers. For this reason the stripper column flow profiles were not constant.

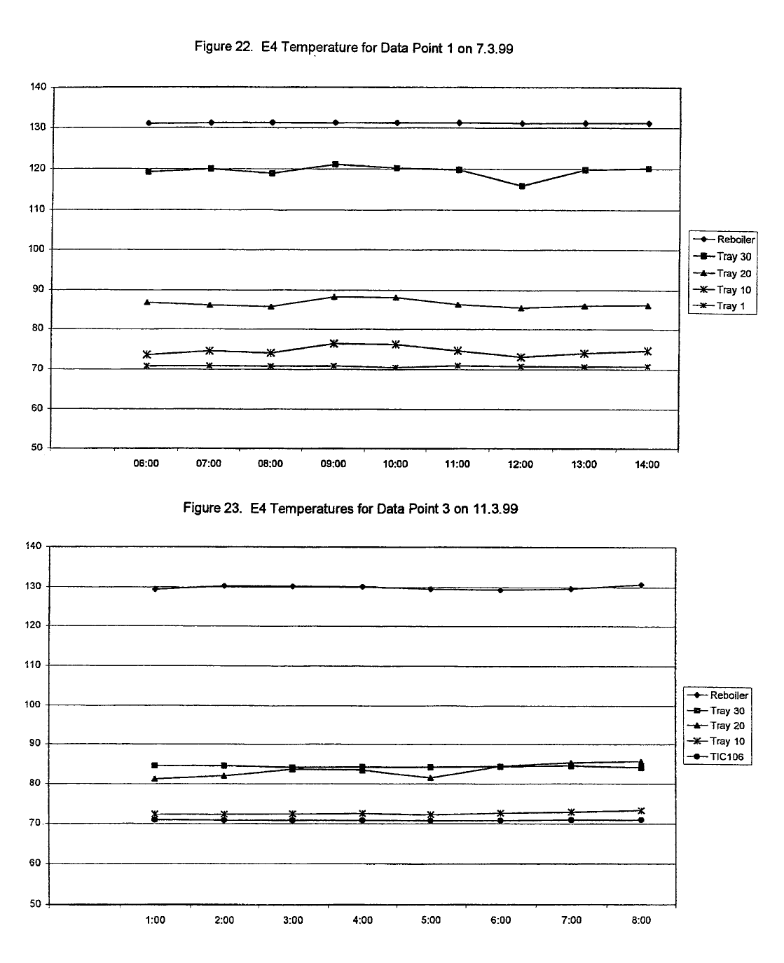

Constant profile temperatures as shown in FIGS. 22 to 25.

Constant analytical results for critical components in product streams.

TABLE 25
Ethanol Content of Azeotropic Column Bottoms

| Data Point 1 | | Data Point 3 | | Data Point 4 | | Data Point 5 | |
|---|---|---|---|---|---|---|---|
| Time | ETOH (ppm) | Time | ETOH (ppm) | Time | ETOH (ppm) | Time | ETOH (ppm) |
| 06:00 | 6.9 | 00:00 | 561.4 | 20:00 | 5.6 | 18:00 | 3.4 |
| 08:00 | 0.0 | 02:00 | 12.9 | 00:00 | 0.0 | 20:00 | 0.0 |
| 10:00 | 26.3 | 04:00 | 41.9 | 02:00 | 23.9 | 23:00 | 35.1 |
| 12:00 | 3.9 | 06:00 | 21.3 | 06:00 | 9.0 | 02:00 | 9.6 |
| 14:00 | 0.0 | 08:00 | 4.3 | 08:00 | 0.0 | 03:00 | 6.8 |

As can be seen in Table 25 the ethanol content for the 8 hours preceding all the data points was stable at low concentrations, and also below specification. The concentration of ethanol in the azeotropic column bottoms for Data Point 5 increased drastically to 2436 ppm, 2 hours after the data point samples were taken.

Similar analytical results for the phases from the phase separator and those of the two recycle containers are shown in Tables 26 to 29.

TABLE 26
Compositions of Azeotropic Column Solvent and
Phase Separator Heavy Phase for Data Point 1

| Stream | 1-Octene | n-Octane | 2-Hexanone | Hexanal | Water | Ethanol |
|---|---|---|---|---|---|---|
| Phase Separator | 12.079 | 1.644 | 0.293 | 0.037 | 9.80 | 67.81 |
| Heavy Phase | | | | | | |
| Azeotropic Column | 13.184 | 1.839 | 0.280 | 0.044 | 9.50 | 66.07 |
| Solvent | | | | | | |

TABLE 27
Compositions of Azeotropic Column Solvent and
Phase Separator Heavy Phase for Data Point 3

| Stream | 1-Octene | n-Octane | 2-Hexanone | Hexanal | Water | Ethanol |
|---|---|---|---|---|---|---|
| Phase Separator | 14.506 | 2.022 | 0.080 | 0.032 | 8.82 | 65.74 |
| Heavy Phase | | | | | | |
| Azeotropic Column | 14.105 | 1.921 | 0.044 | 0.024 | 8.87 | 66.74 |
| Solvent | | | | | | |

TABLE 28
Compositions of Azeotropic column solvent and
Phase Separator Heavy Phase for Data Point 4

| Stream | 1-Octene | n-Octane | 2-Hexanone | Hexanal | Water | Ethanol |
|---|---|---|---|---|---|---|
| Phase Separator | 13.937 | 1.932 | — | — | 9.10 | 67.03 |
| Heavy Phase | | | | | | |
| Azeotropic Column | 13.665 | 1.901 | — | — | 8.60 | 67.94 |
| Solvent | | | | | | |

TABLE 29
Compositions of Azeotropic Column Solvent and
Phase Separator Heavy Phase for Data Point 5

| Stream | 1-Octene | n-Octane | 2-Hexanone | Hexanal | Water | Ethanol |
|---|---|---|---|---|---|---|
| Phase Separator | 14.405 | 2.034 | — | — | 8.84 | 66.52 |
| Heavy Phase | | | | | | |
| Azeotropic Column | 13.695 | 1.923 | — | — | 9.3 | 67.27 |
| Solvent | | | | | | |

Mass Balances within 10% error

Because the measured flow rates are small, a small measurement error can result in a significant mass balance error. The overall plant balance based on average flow rates was within 10% balance. However, it should be noted that the flow rates for the stripper column did fluctuate to maintain constant levels in the recycle containers. This has an effect on the plant balance.

TABLE 30
Data Point Mass Balances based on Average Flow Rates

| Data Point | DP 1 | DP 3 | DP 4 | DP 5 |
|---|---|---|---|---|
| Mass Balance | 96.2% | 103.8% | 92.1% | 89.3% |

Phase separation on the trays in the Azeotropic Column

If the water content of the solvent reflux to the azeotropic column becomes to high, it causes phase separation below the feed point in the azeotropic column. For this reason, the water levels in the reflux were maintained below 10% (below 11.4% on a HC-free basis).

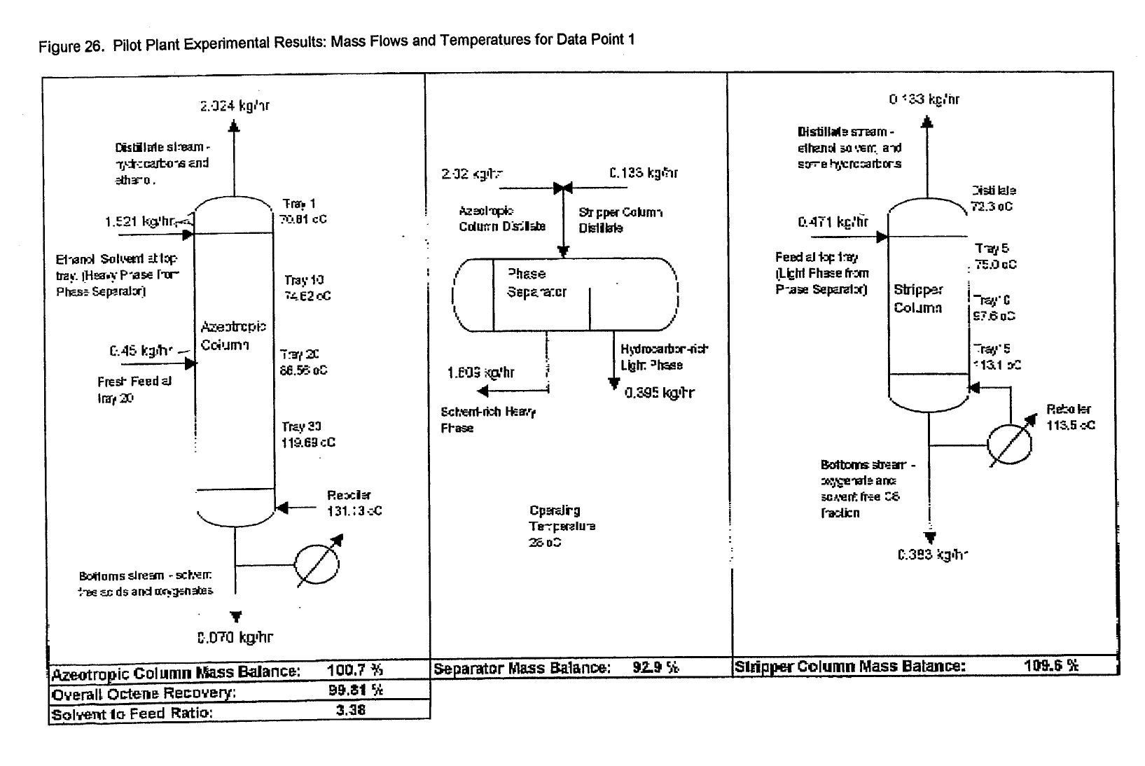

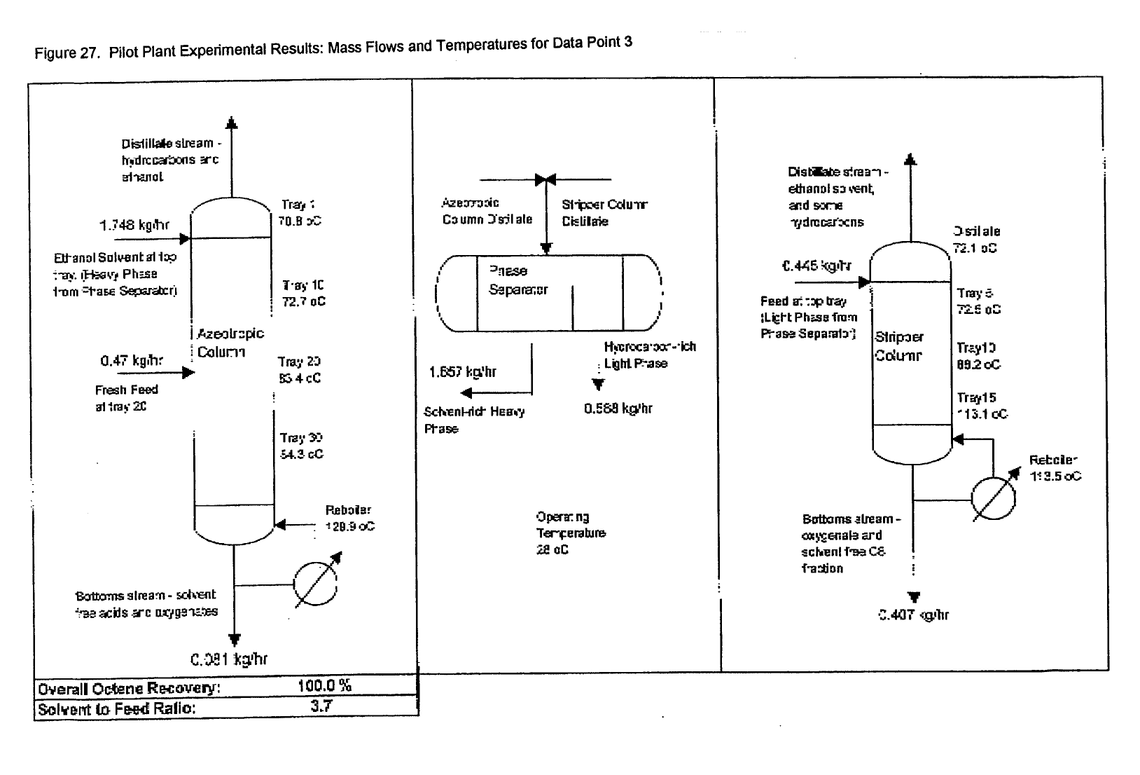

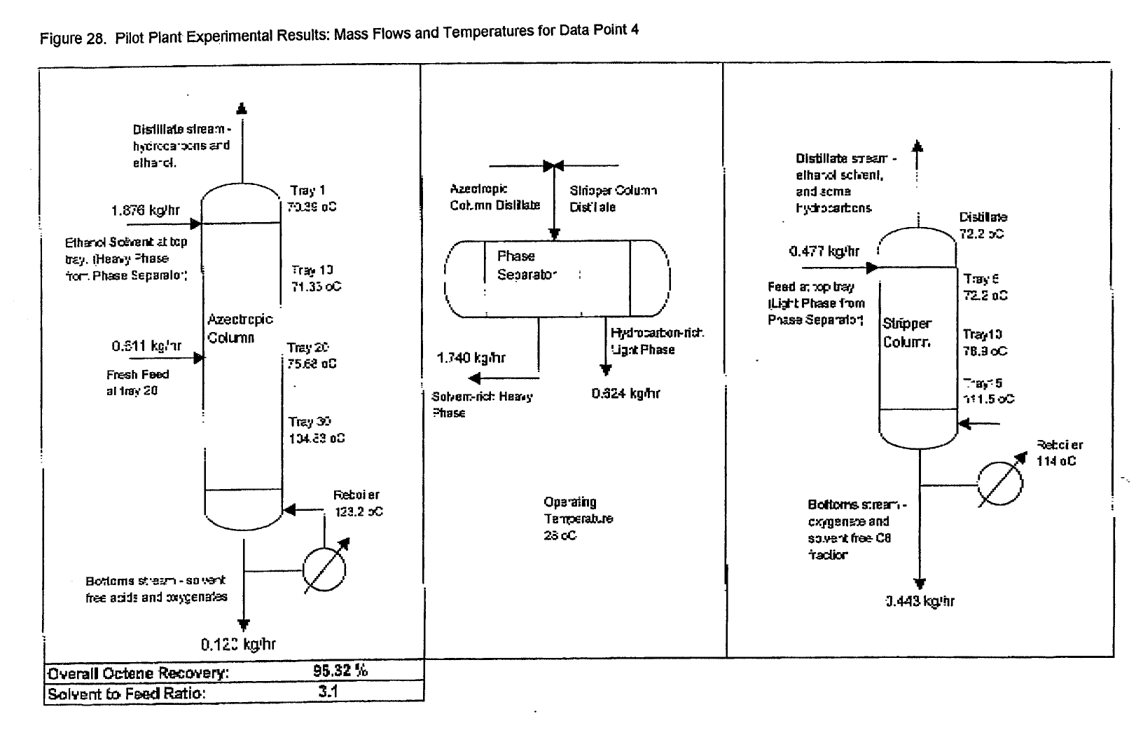

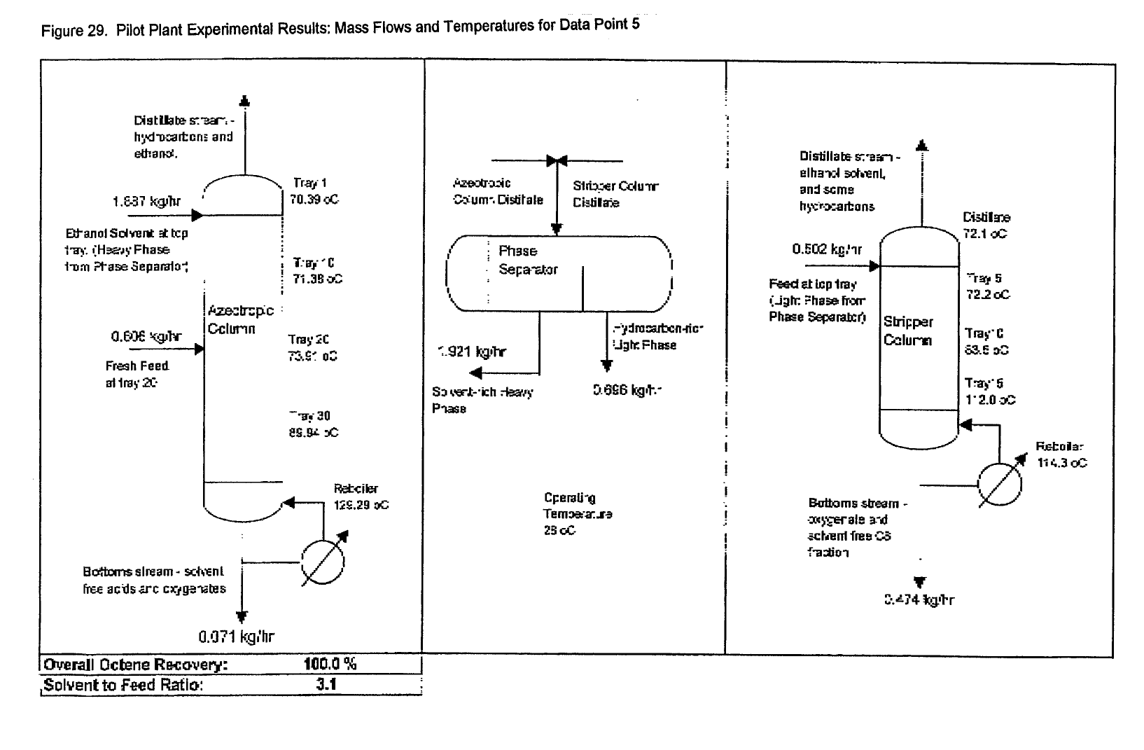

The measured mass flows and temperatures for data points 1, 3, 4, and 5 are presented in FIGS. 26 to 29. The thermocouples for temperature measurement were located between sections, and actually measured the temperature of the liquid from the stage above which they are located. The tray 1 thermocouple in the azeotropic column measured the distillate temperature.

The distillate samples for both the azeotropic and stripper columns phase separate as a result of cooling from process to ambient temperature. Where possible, the results for both phases are presented here in Tables 31 to 42.

TABLE 31
Azeotropic Column (wt %) Data Point 1

| Stream | 1-Octene | n-Octane | 2-Hexanone | Hexanal | Water | Ethanol |
|---|---|---|---|---|---|---|
| Solvent | 13.184 | 1.839 | 0.280 | 0.044 | 9.5 | 66.073 |
| Distillate Light Phase | 49.763 | 8.054 | 0.194 | 0.032 | 0.65 | 11.963 |
| Distillate Heavy Phase | 13.083 | 1.832 | 0.277 | 0.046 | 10.28 | 66.002 |
| Bottoms | 0.600 | 0.132 | 41.297 | 2.032 | n.a. | 0.000 ppm |

TABLE 32
Stripper Column (wt %) Data Point 1

| Stream | 1-Octene | n-Octane | 2-Hexanone | Hexanal | Water | Ethanol |
|---|---|---|---|---|---|---|
| Feed | 49.008 | 7.944 | 0.180 | 0.041 | 0.85 | 14.030 |
| Distillate Light Phase | No Sample | | | | | |
| Distillate Heavy Phase | 19.171 | 2.891 | 0.033 | — | 4.49 | 57.076 |
| Bottoms | 57.923 | 9.437 | 0.217 | 0.057 | n.a. | 0.000 ppm |

TABLE 33
Phase Separator (wt %) Data Point 1

| Stream | 1-Octene | n-Octane | 2-Hexanone | Hexanal | Water | Ethanol |
|---|---|---|---|---|---|---|
| Heavy Phase | 12.079 | 1.644 | 0.293 | 0.037 | 9.8 | 67.813 |
| Light Phase * | 48.930 | 8.120 | 0.201 | 0.047 | 0.4 | 11.178 |

TABLE 34
Azeotropic Column (wt %) Data Point 3

| | 1- | n- | 2- | | | |
|---|---|---|---|---|---|---|
| Stream | Octene | Octane | Hexanone | Hexanal | Water | Ethanol |
| Solvent | 14.105 | 1.921 | 0.044 | 0.024 | 8.87 | 66.740 |
| Distillate | 52.041 | 8.504 | 0.068 | 0.034 | 0.4 | 11.096 |
| Light | | | | | | |
| Phase | | | | | | |
| Distillate | 14.125 | 2.001 | 0.002 | 0.008 | 9.10 | 65.564 |
| Heavy | | | | | | |
| Phase | | | | | | |
| * | | | | | | |
| Bottoms | 0.000 | 0.000 | 43.423 | 2.061 | n.a. | 4.31 ppm |

TABLE 35
Stripper Column (wt %) Data Point 3

| | 1- | n- | 2- | | | |
|---|---|---|---|---|---|---|
| Stream | Octene | Octane | Hexanone | Hexanal | Water | Ethanol |
| Feed | 51.735 | 8.341 | 0.030 | 0.021 | 0.4 | 12.148 |
| Distillate | No Sample | | | | | |
| Light | | | | | | |
| Phase | | | | | | |
| Distillate | 18.9444 | 2.186 | — | — | 3.56 | 59.086 |
| Heavy | | | | | | |
| Phase | | | | | | |
| Bottoms | 60.049 | 9.730 | 0.028 | 0.019 | n.a. | 0.000 |
| | | | | | | ppm |

TABLE 36
Phase Separator (wt %) Data Point 3

| | 1- | n- | 2- | | | |
|---|---|---|---|---|---|---|
| Stream | Octene | Octane | Hexanone | Hexanal | Water | Ethanol |
| Heavy | 14.506 | 2.022 | 0.080 | 0.032 | 8.82 | 65.744 |
| Phase | | | | | | |
| Light | 50.700 | 8.268 | 0.049 | 0.028 | 0.1 | 13.034 |
| Phase | | | | | | |

TABLE 37
Azeotropic Column (wt %) Data Point 4

| | 1- | n- | 2- | | | |
|---|---|---|---|---|---|---|
| Stream | Octene | Octane | Hexanone | Hexanal | Water | Ethanol |
| Solvent | 13.665 | 1.901 | — | — | 8.6 | 67.937 |
| Distillate | 54.471 | 8.431 | — | — | 0.49 | 11.983 |
| Light | | | | | | |
| Phase | | | | | | |
| Distillate | 13.284 | 1.835 | — | — | 9.39 | 67.915 |
| Heavy | | | | | | |
| Phase | | | | | | |
| Bottoms | 10.862 | 2.105 | 32.892 | 2.085 | n.a. | 0.000 |
| | | | | | | ppm |

TABLE 38
Stripper Column (wt %) Data Point 4

| | 1- | n- | 2- | | | |
|---|---|---|---|---|---|---|
| Stream | Octene | Octane | Hexanone | Hexanal | Water | Ethanol |
| Feed * | 48.218 | 7.955 | — | — | 0.4 | 15.877 |
| Distillate | No Sample | | | | | |
| Light | | | | | | |
| Phase | | | | | | |
| Distillate | 14.151 | 1.956 | — | — | 3.48 | 72.345 |
| Heavy | | | | | | |
| Phase | | | | | | |
| Bottoms | 59.880 | 9.785 | — | — | n.a. | 0.000 |
| | | | | | | ppm |

TABLE 39
Phase Separator (wt %) Data Point 4

| | 1- | n- | 2- | | | |
|---|---|---|---|---|---|---|
| Stream | Octene | Octane | Hexanone | Hexanal | Water | Ethanol |
| Heavy | 13.937 | 1.932 | — | — | 9.1 | 67.034 |
| Phase | | | | | | |
| Light | 48.575 | 7.892 | — | — | 0.4 | 16.041 |
| Phase * | | | | | | |

TABLE 40
Azeotropic Column (wt %) Data Point 5

| | 1- | n- | 2- | | | |
|---|---|---|---|---|---|---|
| Stream | Octene | Octane | Hexanone | Hexanal | Water | Ethanol |
| Solvent | 13.695 | 1.923 | — | — | 9.3 | 67.272 |
| Distillate | 50.924 | 8.294 | — | — | 0.49 | 14.178 |
| Light | | | | | | |
| Phase | | | | | | |
| Distillate | 13.357 | 1.867 | — | — | 9.98 | 69.119 |
| Heavy | | | | | | |
| Phase | | | | | | |
| Bottoms | 0.000 | 0.000 | 41.644 | 2.320 | n.a. | 6.8 ppm |

TABLE 41
Stripper Column (wt %) Data Point 5

| | 1- | n- | 2- | | | |
|---|---|---|---|---|---|---|
| Stream | Octene | Octane | Hexanone | Hexanal | Water | Ethanol |
| Feed * | 47.481 | 7.766 | — | — | 0.7 | 16.977 |
| Distillate | No Sample | | | | | |
| Light | | | | | | |
| Phase | | | | | | |
| Distillate | 19.096 | 2.822 | 0.017 | — | 3.48 | 59.588 |
| Heavy | | | | | | |
| Phase | | | | | | |
| Bottoms | 59.856 | 9.934 | — | — | n.a. | 18.9 ppm |

TABLE 42
Phase Separator (wt %) Data Point 5

| | 1- | n- | 2- | | | |
|---|---|---|---|---|---|---|
| Stream | Octene | Octane | Hexanone | Hexanal | Water | Ethanol |
| Heavy | 14.405 | 2.034 | — | — | 8.84 | 66.521 |
| Phase | | | | | | |
| Light | 46.314 | 7.577 | — | — | 0.5 | 18.848 |
| Phase * | | | | | | |

Symbol: ‘-’, Status: undetected components on GC results

Symbol: ‘n.a.’, Status: no analysis done

Symbol: ‘ ’, Status: Water analysis done 2 months after sampling

use as an indication of water content.

Symbol: ‘*’, Status: Sample re-analysed 2 months later due to misleading analytical results. This analysis was also done on an FFAP column, but with N₂ carrier gas. The 2-Hexanone and 1-Hexanal components are not as easily separated. Use these results as an indication of stream composition.

The feed composition, i.e. stream 22, was as per Example 1.

Mass Balances and Product Compositions

Azeotropic Column 42:

The feed and reflux flow rates to the azeotropic column were measured on scales. The overheads flow was not measured, and the bottoms flow was very dependent on the level in the reboiler. Therefore it was assumed that the measured azeotropic column reflux and feed flow rates were reliable.

The theoretical number of stages and mass split were calculated to match the bottoms 1-octene and 2-hexanone compositions with experimental data. The feed position was selected to match the 1-octene, n-octane and 2-hexanone liquid composition profiles, while maintaining the match on the bottoms composition.

Stripper Column 44:

Using the simulation results obtained for the azeotropic column as basis, an overall plant mass balance was calculated. This fixed the stripper column bottoms flow. The number of theoretical stages was fixed at eight. The feed flow rate to the stripper was calculated to match the bottoms 1-octene and n-octane experimental data.

A comparison between measured and simulated mass flow rates is presented in tables 43 to 46. The process was simulated at scaled up flow rates (tons instead of kilograms), to assist conversion. The simulated flow rates presented here are scaled down to kilograms.

TABLE 43
Mass Flow Rates for Data Point 1

| | | Simulation |
|---|---|---|
| Stream | Measured (kg/hr) | (kg/hr) |
| Azeotropic Column Feed | 0.450 | 0.450 |
| Azeotropic Column Solvent | 1.521 | 1.521 |
| Azeotropic Column Distillate | 2.024 | 1.911 |
| Azeotropic Column Bottoms | 0.070 | 0.060 |
| Stripper Column Feed | 0.471 | 0.5259 |
| Stripper Column Distillate | 0.133 | 0.1359 |
| Stripper Column Bottoms | 0.383 | 0.390 |
| Azeotropic Column Mass Balance | 100.7 | 100.0 |
| Stripper Column Mass Balance | 109.6 | 100.0 |
| Overall System Mass Balance | 96.2 | 100.0 |

TABLE 44
Mass Flow Rates for Data Point 3

| | | Simulation |
|---|---|---|
| Stream | Measured (kg/hr) | (kg/hr) |
| Azeotropic Column Feed | 0.470 | 0.470 |
| Azeotropic Column Solvent | 1.748 | 1.748 |
| Azeotropic Column Bottoms | 0.081 | 0.0645 |
| Stripper Column Feed | 0.445 | 0.5196 |
| Stripper Column Bottoms | 0.407 | 0.4055 |
| Overall System Mass Balance | 103.8 | 100.0 |

TABLE 45
Mass Flow Rates for Data Point 4

| | | Simulation |
|---|---|---|
| Stream | Measured (kg/hr) | (kg/hr) |
| Azeotropic Column Feed | 0.611 | 0.611 |
| Azeotropic Column Solvent | 1.876 | 1.876 |
| Azeotropic Column Bottoms | 0.120 | 0.1141 |
| Stripper Column Feed | 0.477 | 0.6915 |
| Stripper Column Bottoms | 0.443 | 0.4969 |
| Overall System Mass Balance | 92.1 | 100.0 |

TABLE 46
Mass Flow Rates for Data Point 5

| | | Simulation |
|---|---|---|
| Stream | Measured (kg/hr) | (kg/hr) |
| Azeotropic Column Feed | 0.606 | 0.606 |
| Azeotropic Column Solvent | 1.887 | 1.887 |
| Azeotropic Column Bottoms | 0.071 | 0.0846 |
| Stripper Column Feed | 0.502 | 0.7493 |
| Stripper Column Bottoms | 0.474 | 0.5214 |
| Overall System Mass Balance | 89.2 | 100.0 |

The material balances as shown in tables 43 to 46 were used for the simulations. The simulations were performed on Aspen Plus™ using the Unifac Dortmund group contribution method to predict the vapour-liquid and liquid-liquid equilibrium data. The azeotropic column was also simulated with only vapour and liquid as valid phases, to assist simulation convergence. It is possible that two liquid phases exist in this column, but care was taken to ensure that only 1 liquid phase was present during data point sampling.

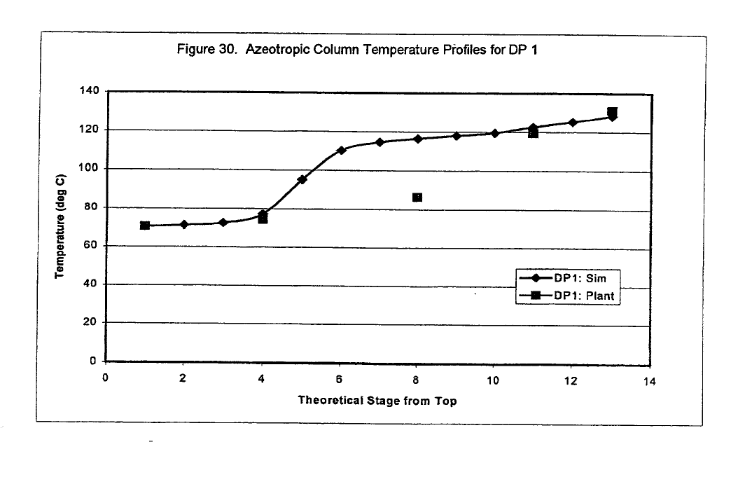

In FIGS. 30 and 32 the plant temperature profiles were lower than the predicted temperature profiles. This is because the bottoms from the azeotropic column contained C₈'s, and no solvent, causing the simulation predicts a “hot profile” solution for these two data points. In FIG. 30, it is only at the feed point that there is a large temperature difference.

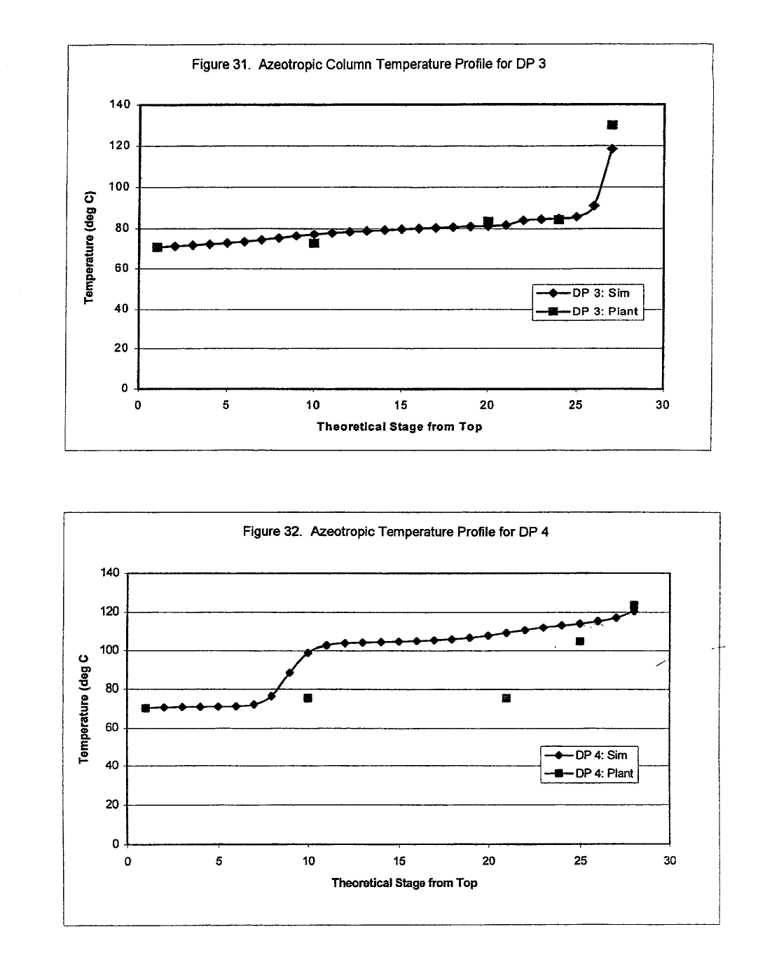

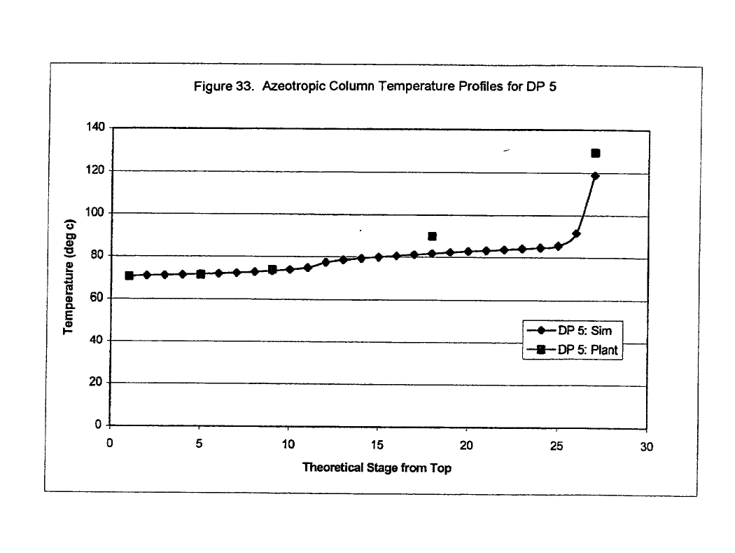

The predicted profiles for data points 3 and 5 (FIGS. 31 and 33), in which there were no C₈-components in the column bottoms, match the plant data well.

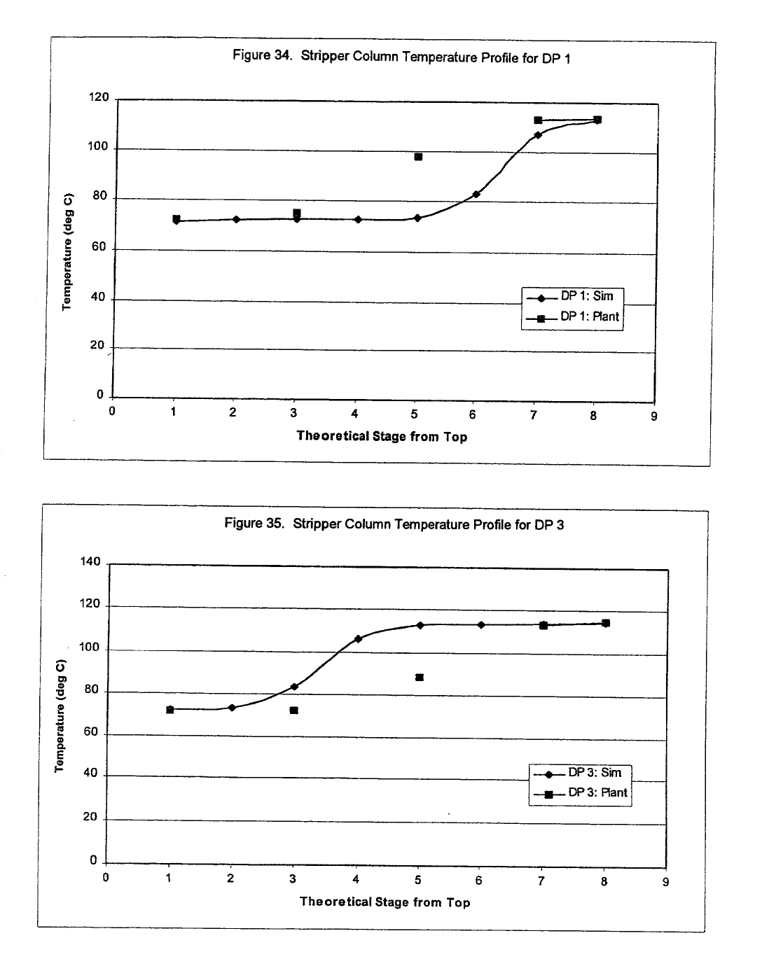

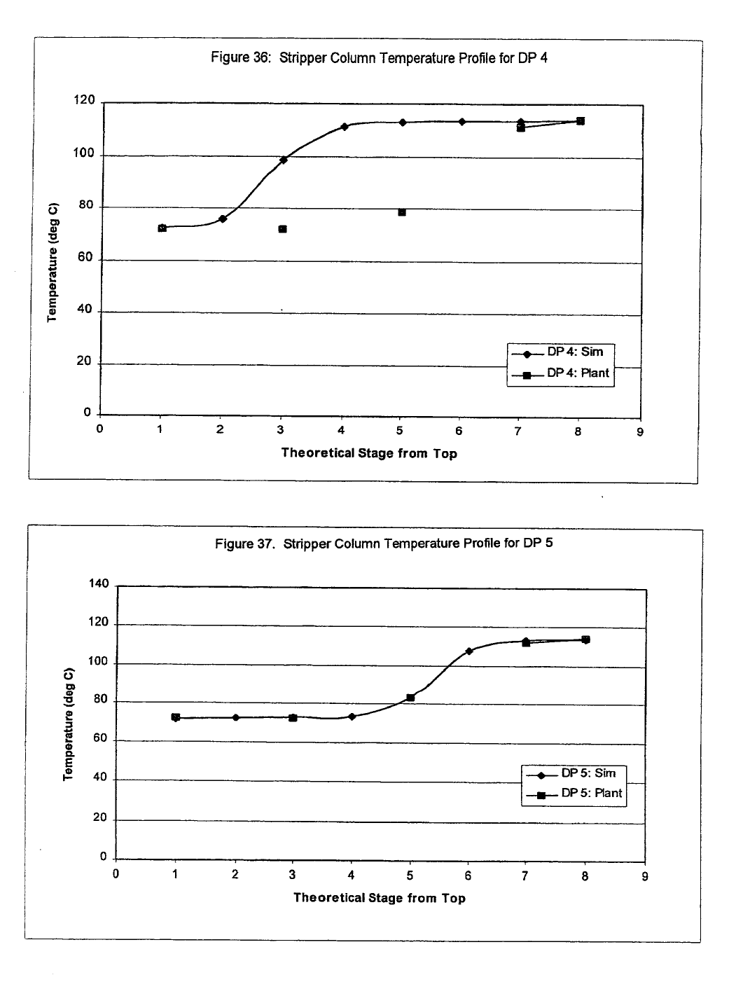

The stripper column temperature profiles for data points 1, 3, 4 and 5 are shown in FIGS. 34 to 37. The only profile with a good match is that of data point 5. For Data Point 1, the simulations predicted a colder profile. For Data Points 3 and 4, the simulation predicted a hotter profile. This could also be related to ethanol levels in the column. The predicted levels of ethanol in the bottoms for Data Point 1 are higher than for the other data points (OOM E-4). For data points 3 and 4, the simulation predicted very low levels of ethanol in the stripper bottoms (OOM E-7).

Samples were taken from sampling points between the sections of the azeotropic column, with the purpose of examining the liquid composition profiles.

The profile samples for data point 4 were taken a few hours after the product data point samples. The average mass flow rates, and temperatures for the column had changed by this stage. No recycle and bottoms samples were taken at this time and the profiles were simulated at the same conditions as for the data point.

Profile samples could only be taken above the feed point. The sample points were located between column sections, and the liquid samples were of the tray above the sample point.

The simulation of data point 4 profiles yielded the best results (in terms of 1-octene, n-octane and 2-hexanone bottoms concentration) for an azeotropic column with 28 stages, and the feed reporting to stage 22. For data point 5, the optimum was a column with 27 stages, and feed stage 10.

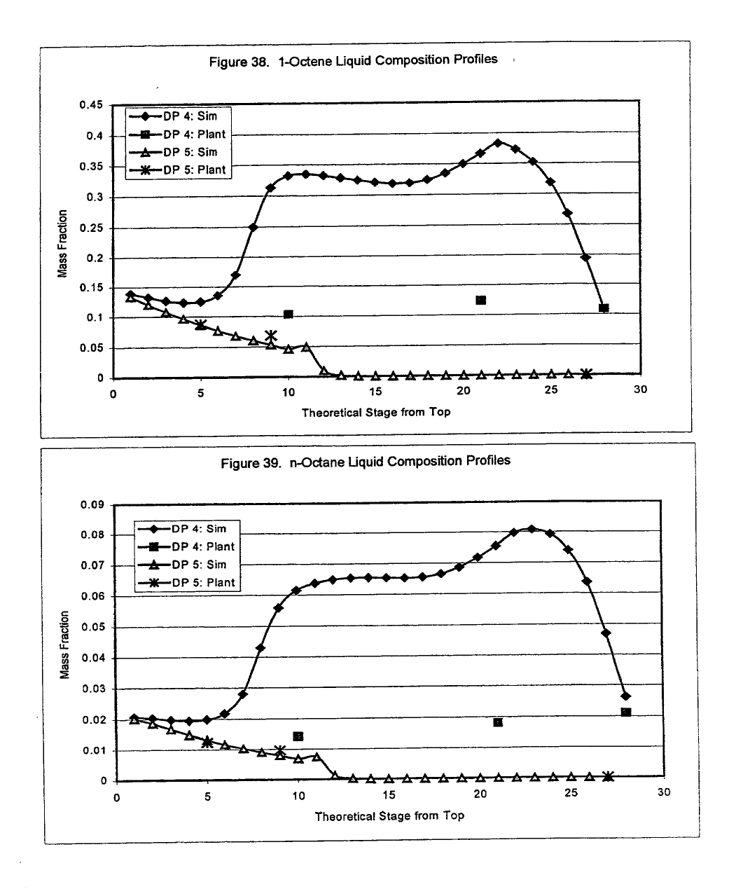

There is a marked similarity between the 1-Octene and n-Octane profiles of FIGS. 38 and 39. For both components, the predicted profile of data point 5 matches the experimental results well.

The same feed composition was used for all data point simulations. The GC-results for the solvent recycle to the azeotropic column, and the feed to stripper column was used as input to the simulation. Manipulated column parameters include bottoms flow rates, theoretical number of stages, and feed stage.

The total C₆-component concentration is determined by combining the 2-hexanone and 1-hexanal concentrations. This compensates for integration errors that result because of their close proximity on the GC-traces.

Azeotropic Column Bottoms 58:

For all data points, there is a good match for the azeotropic column bottoms 1-octene and n-octane concentration results (tables 47, 51, 55 and 59). This is because column parameters were manipulated to obtain a good match for these two components. The corresponding predicted total C₆-component concentrations also match the experimental data well. For data points 3 and 5, where a “cold profile” simulation result is required to predict zero concentrations of C₈'s, the predicted solvent concentrations in the bottoms are higher than the plant results. All the simulations predict higher acid concentrations in the bottoms than determined experimentally.

Stripper Column Bottoms 57:

The measured and simulated data for the stripper column bottoms compares very well for all data points (tables 49, 53, 57 and 61). There is a good match for the 1-octene and n-octane concentration results. Once again column parameters were manipulated to obtain a good match for these two components. There is also good agreement for the toluene, 2-hexanone and hexanal results.

Azeotropic Column Distillate 46:

There is a reasonably good agreement between measured and simulated data for the azeotropic column distillate streams. In both the light and heavy phases, the concentrations of 1-octene compare particularly well. There is also good agreement between the predicted and measured n-octane concentrations. The simulation predicts considerably less ethanol in the light phase than was measured. In the heavy phase, the simulation predicts comparable water and ethanol concentrations.

Stripper Column Distillate 48:

The simulations often predicted the existence of only a light phase, while there are no light phase samples available from the plant to be analyzed. For data point 1, there is good agreement between the plant and simulated data for the heavy phase. The data point 3 heavy phase distillate sample from the plant compares reasonably well with the predicted light phase of the stripper column distillate. For data point 4 however, the simulated light phase contains significantly more C₈'s and less ethanol, than was present in the plant sample of the distillate heavy phase.

TABLE 47
Azeotropic Column Results for Data Point 1

| | Input | | Results | Plant Data |
|---|---|---|---|---|
| Component | Feed | Solvent | Bottoms | Bottoms |
| Toluene | 0.902 | 0.533 | 0.023 | 0.037 |
| 1-Octene | 51.166 | 13.184 | 0.575 | 0.600 |
| n-Octane | 8.432 | 1.839 | 0.081 | 0.132 |
| Ethyl Benzene | 0.108 | 0.034 | 0.298 | 0.193 |
| Butyl Acetate | 0.075 | 0.030 | 0.542 | 0.207 |
| 2-Hexanone | 5.840 | 0.280 | 38.960 | 41.297 |
| Hexanal | 0.507 | 0.044 | 4.262 | 2.032 |
| 1-Butanol | 0.076 | 0.000 | 0.123 | 0.167 |
| 1-Pentanol | 2.802 | 0.000 | 21.010 | 16.949 |
| Propanoic Acid | 0.992 | 0.000 | 7.436 | 3.820 |
| Isobutanoic Acid | 0.775 | 0.000 | 5.811 | 4.062 |
| Butanoic Acid | 0.077 | 0.000 | 0.577 | 0.313 |
| Water | 0.200 | 9.500 | 0.000 | n.a. |
| Ethanol | 0.000 | 66.073 | 0.0 ppm | 0.0 ppm |
| Total C₆ (mass %) | 0.000 | 0.000 | 43.222 | 43.329 |
| Flow Rate (kg/hr) | 450 | 1521 | 60.00 | 70 |
| | | | | (equiv- |
| | | | | alent) |
| Temperature (° C.) | 105 | 55 | 128.29 | 131.13 |

| | |
|---|---|
| Theoretical Stages | 13 |
| Feed Stage | 10 |

TABLE 48
Azeotropic Column Distillate for Data Point 1

| | Simulation | Heavy | Heavy | Light | Light |
|---|---|---|---|---|---|
| | Results for | Phase | Phase | Phase | Phase |
| | Total | Simulation | Plant | Simulation | Plant |
| Component | Distillate | Result | Data | Result | Data |
| Toluene | 0.636 | 0.456 | 0.504 | 1.171 | 0.194 |
| 1-Octene | 22.524 | 12.694 | 13.083 | 51.764 | 49.763 |
| n-Octane | 3.447 | 1.404 | 1.833 | 9.524 | 8.054 |
| 2-Hexanone | 0.375 | 0.430 | 0.277 | 0.209 | 0.194 |
| Hexanal | 0.021 | 0.023 | 0.046 | 0.014 | 0.032 |
| Water | 7.608 | 10.070 | 10.280 | 0.285 | 0.650 |
| Ethanol | 52.589 | 68.509 | 66.002 | 5.233 | 11.963 |
| Flow Rate | 1911.00 | 1430.20 | | 480.80 | |
| (kg/hr) | | | | | |
| Temperature | 70.73 | 28 | | 28 | |
| (° C.) | | | | | |

TABLE 49
Stripper Column Results for Data Point 1

| | Input | Simulation Result | Plant Data |
|---|---|---|---|
| Component | Feed | Bottoms | Bottoms |
| Toluene | 1.030 | 1.130 | 1.216 |
| 1-Octene | 49.008 | 57.965 | 57.923 |
| n-Octane | 7.944 | 9.516 | 9.437 |
| 2-Hexanone | 0.180 | 0.220 | 0.217 |
| Hexanal | 0.041 | 0.051 | 0.057 |
| Water | 0.850 | 0.000 | n.a. |
| Ethanol | 14.030 | 360 ppm | 0.0 ppm |
| Flow Rate (kg/hr) | 525.9 | 390 | 383 (equivalent) |
| Temperature (° C.) | 50 | 113.32 | 113.5 |

| | |
|---|---|
| Theoretical Stages | 8 |

TABLE 50
Stripper Column Distillate for Data Point 1

| | Simulation | Light | Light | Heavy | Heavy |
|---|---|---|---|---|---|
| | Results for | Phase | Phase | Phase | Phase |
| | Total | Simulation | Plant | Simulation | Plant |
| Component | Distillate | Result | Data | Result | Data |
| Toluene | 0.744 | 1.108 | No Sample | 0.696 | 0.726 |
| 1-Octene | 23.306 | 47.829 | | 20.090 | 19.171 |
| n-Octane | 3.431 | 8.357 | | 2.785 | 2.891 |
| 2-Hexanone | 0.064 | 0.039 | | 0.067 | 0.032 |
| Hexanal | 0.012 | 0.007 | | 0.012 | — |
| Water | 3.289 | 0.295 | | 3.682 | 4.490 |
| Ethanol | 54.192 | 9.747 | | 60.022 | 57.076 |
| Flow Rate | 135.9 | 15.76 | | 120.14 | |
| (kg/hr) | | | | | |
| Temperature | 71.64 | 28 | | 28 | |
| (° C.) | | | | | |

TABLE 51
Azeotropic Column Results for Data Point 3

| | Input | | Results | Plant Data |
|---|---|---|---|---|
| Component | Feed | Solvent | Bottoms | Bottoms |
| Toluene | 0.902 | 0.477 | 0.000 | 0.065 |
| 1-Octene | 51.166 | 14.105 | 0.000 | — |
| n-Octane | 8.432 | 1.921 | 0.000 | — |
| Ethyl Benzene | 0.108 | 0.036 | 0.000 | 0.158 |
| Butyl Acetate | 0.075 | 0.008 | 0.043 | 0.299 |
| 2-Hexanone | 5.840 | 0.044 | 43.209 | 43.423 |
| Hexanal | 0.507 | 0.024 | 3.395 | 2.062 |
| 1-Butanol | 0.076 | 0.000 | 0.555 | 0.234 |
| 1-Pentanol | 2.802 | 0.000 | 20.421 | 17.151 |
| Propanoic Acid | 0.992 | 0.000 | 7.226 | 3.722 |
| Isobutanoic Acid | 0.775 | 0.000 | 5.646 | 3.946 |
| Butanoic Acid | 0.077 | 0.000 | 0.561 | 0.291 |
| Water | 0.200 | 8.870 | 0.735 | n.a. |
| Ethanol | 0.000 | 66.740 | 75 ppm | 4.3 ppm |
| Total C₆ (mass %) | 0.000 | 0.000 | 46.604 | 45.485 |
| Flow Rate (kg/hr) | 470 | 1748 | 64.50 | 81 (equivalent) |
| Temperature (° C.) | 105 | 55 | 118.50 | 129.9 |

| | |
|---|---|
| Theoretical Stages | 27 |
| Feed Stage | 21 |

TABLE 52
Azeotropic Column Distillate for Data Point 3

| | Simulation | Heavy | Heavy | Light | Light |
|---|---|---|---|---|---|
| | Results for | Phase | Phase | Phase | Phase |
| | Total | Simulation | Plant | Simulation | Plant |
| Component | Distillate | Result | Data * | Result | Data |
| Toluene | 0.584 | 0.441 | 0.476 | 1.072 | 0.928 |
| 1-Octene | 22.616 | 13.712 | 14.125 | 52.963 | 52.041 |
| n-Octane | 3.399 | 1.555 | 2.000 | 9.687 | 8.504 |
| 2-Hexanone | 0.016 | 0.018 | 0.002 | 0.009 | 0.068 |
| Hexanal | 0.028 | 0.031 | 0.083 | 0.018 | 0.034 |
| Water | 7.221 | 9.257 | 9.100 | 0.282 | 0.400 |
| Ethanol | 54.173 | 68.458 | 65.564 | 5.481 | 11.096 |
| Flow Rate | 2153.5 | 1655 | | 488.5 | |
| (kg/hr) | | | | | |
| Temperature | 70.70 | 28 | | 28 | |
| (° C.) | | | | | |

TABLE 53
Stripper Column Results for Data Point 3

| | Input | Simulation Result | Plant Data |
|---|---|---|---|
| Component | Feed | Bottoms | Bottoms |
| Toluene | 0.910 | 0.975 | 0.949 |
| 1-Octene | 51.735 | 59.505 | 60.049 |
| n-Octane | 8.341 | 9.698 | 9.730 |
| 2-Hexanone | 0.030 | 0.036 | 0.028 |
| Hexanal | 0.021 | 0.025 | 0.019 |
| Water | 0.400 | 0.000 | n.a. |
| Ethanol | 12.148 | 0.0 ppm | 0.0 ppm |
| Flow Rate (kg/hr) | 519.6 | 405.5 | 407 (equivalent) |
| Temperature (° C.) | 50 | 114.07 | 113.5 |

| | |
|---|---|
| Theoretical Stages | 8 |

TABLE 54
Stripper Column Distillate for Data Point 3

| | Simulation | Simulation | Heavy | Heavy |
|---|---|---|---|---|
| | Results for | Result - | Phase | Phase |
| | Total | only one | Plant | Simulation |
| Component | Distillate | phase | Data | Result |
| Toluene | 0.680 | 0.680 | 0.590 | No Heavy |
| 1-Octene | 24.119 | 24.119 | 18.944 | Phase |
| n-Octane | 3.518 | 3.518 | 2.186 | Predicted |
| 2-Hexanone | 0.012 | 0.012 | — | |
| Hexanal | 0.007 | 0.007 | — | |
| Water | 1.822 | 1.822 | 3.560 | |
| Ethanol | 55.320 | 55.320 | 59.086 | |
| Flow Rate (kg/hr) | 114.1 | 114.1 | | |
| Temperature (° C.) | 72.58 | 28 | | |

TABLE 55
Azeotropic Column Results for Data Point 4

| | Input | | Results | Plant Data |
|---|---|---|---|---|
| Component | Feed | Solvent | Bottoms | Bottoms |
| Toluene | 0.902 | 0.473 | 0.054 | 1.511 |
| 1-Octene | 51.166 | 13.665 | 10.810 | 10.862 |
| n-Octane | 8.432 | 1.901 | 2.623 | 2.105 |
| Ethyl Benzene | 0.108 | 0.000 | 0.575 | 0.169 |
| Butyl Acetate | 0.075 | 0.000 | 0.402 | 0.319 |
| 2-Hexanone | 5.840 | 0.000 | 31.271 | 32.892 |
| Hexanal | 0.507 | 0.000 | 2.715 | 2.085 |
| 1-Butanol | 0.076 | 0.000 | 0.406 | 0.103 |
| 1-Pentanol | 2.802 | 0.000 | 15.006 | 15.140 |
| Propanoic Acid | 0.992 | 0.000 | 5.310 | 3.622 |
| Isobutanoic Acid | 0.775 | 0.000 | 4.149 | 0.035 |
| Butanoic Acid | 0.077 | 0.000 | 0.412 | 0.274 |
| Water | 0.200 | 8.600 | 0.000 | n.a. |
| Ethanol | 0.000 | 67.937 | 0.0 ppm | 0.0 ppm |
| Total C₆ (mass %) | 0.000 | 0.000 | 33.986 | 34.977 |
| Flow Rate (kg/hr) | 611 | 1876 | 114.10 | 120 (equivalent) |
| Temperature (° C.) | 105 | 55 | 120.68 | 123.2 |

| | |
|---|---|
| Theoretical Stages | 28 |
| Feed Stage | 22 |

TABLE 56
Azeotropic Column Distillate for Data Point 4

| | Simulation | Heavy | Heavy | Light | Light |
|---|---|---|---|---|---|
| | Results for | Phase | Phase | Phase | Phase |
| | Total | Simulation | Plant | Simulation | Plant |
| Component | Distillate | Result | Data | Result | Data |
| Toluene | 0.604 | 0.459 | 0.471 | 1.083 | 0.961 |
| 1-Octene | 23.458 | 14.307 | 13.284 | 53.721 | 54.472 |
| n-Octane | 3.548 | 1.650 | 1.836 | 9.826 | 8.431 |
| 2-Hexanone | 0.000 | 0.000 | — | 0.000 | — |
| Hexanal | 0.000 | 0.000 | — | 0.000 | — |
| Water | 6.851 | 8.837 | 9.390 | 0.281 | 0.490 |
| Ethanol | 53.711 | 68.261 | 67.915 | 5.597 | 11.983 |
| Flow Rate | 2372.90 | 1821.93 | | 550.97 | |
| (kg/hr) | | | | | |
| Temperature | 70.56 | 28.00 | | 28.00 | |
| (° C.) | | | | | |

TABLE 57
Stripper Column Results for Data Point 4

| | Input | Simulation Result | Plant Data |
|---|---|---|---|
| Component | Feed | Bottoms | Bottoms |
| Toluene | 0.935 | 0.999 | 1.081 |
| 1-Octene | 48.025 | 57.863 | 59.880 |
| n-Octane | 7.923 | 9.694 | 9.785 |
| 2-Hexanone | 0.000 | 0.000 | — |
| Hexanal | 0.000 | 0.000 | — |
| Water | 0.400 | 0.000 | n.a. |
| Ethanol | 15.813 | 0.0 ppm | 0.0 ppm |
| Flow Rate (kg/hr) | 691.5 | 496.9 | 443 (equivalent) |
| Temperature (° C.) | 50 | 114.16 | 114.0 |

| | |
|---|---|
| Theoretical Stages | 8 |

TABLE 58
Stripper Column Distillate for Data Point 4

| | Simulation | Simulation | Light | Heavy | Heavy |
|---|---|---|---|---|---|
| | Results for | Result - | Phase | Phase | Phase |
| | Total | only one | Plant | Simulation | Plant |
| Component | Distillate | phase | Data | Result | Data |
| Toluene | 0.733 | 0.733 | No Sample | No Heavy | 0.502 |
| 1-Octene | 22.906 | 22.906 | | Phase | 14.151 |
| n-Octane | 3.402 | 3.402 | | Predicted | 1.956 |
| 2-Hexanone | 0.000 | 0.000 | | | — |
| Hexanal | 0.000 | 0.000 | | | — |
| Water | 1.421 | 1.421 | | | 3.480 |
| Ethanol | 56.191 | 56.191 | | | 72.345 |
| Flow Rate | 194.6 | 194.6 | | | |
| (kg/hr) | | | | | |
| Temperature | 72.51 | 28 | | | |
| (° C.) | | | | | |

TABLE 59
Azeotropic Column Results for Data Point 5

| | Input | | Results | Plant Data |
|---|---|---|---|---|
| Component | Feed | Solvent | Bottoms | Bottoms |
| Toluene | 0.902 | 0.457 | 0.000 | 1.820 |
| 1-Octene | 51.166 | 13.695 | 0.000 | — |
| n-Octane | 8.432 | 1.923 | 0.000 | — |
| Ethyl Benzene | 0.108 | 0.038 | 0.000 | 0.330 |
| Butyl Acetate | 0.075 | 0.000 | 0.436 | 0.375 |
| 2-Hexanone | 5.840 | 0.000 | 41.828 | 41.644 |
| Hexanal | 0.507 | 0.000 | 3.629 | 2.320 |
| 1-Butanol | 0.076 | 0.000 | 0.546 | 0.187 |
| 1-Pentanol | 2.802 | 0.000 | 20.074 | 16.346 |
| Propanoic Acid | 0.992 | 0.000 | 7.103 | 4.341 |
| Isobutanoic Acid | 0.775 | 0.000 | 5.550 | 4.549 |
| Butanoic Acid | 0.077 | 0.000 | 0.551 | 0.349 |
| Water | 0.200 | 9.200 | 0.683 | n.a. |
| Ethanol | 0.000 | 67.272 | 365 ppm | 6.8 ppm |
| Total C₆ (mass %) | 0.000 | 0.000 | 45.457 | 43.965 |
| Flow Rate (kg/hr) | 606 | 1887 | 84.60 | 71 (equivalent) |
| Temperature (° C.) | 105 | 55 | 118.79 | 129.29 |

| | |
|---|---|
| Theoretical Stages | 27 |
| Feed Stage | 10 |

TABLE 60
Azeotropic Column Distillate for Data Point 5

| | Simulation | Heavy | Heavy | Light | Light |
|---|---|---|---|---|---|
| | Results for | Phase | Phase | Phase | Phase |
| | Total | Simulation | Plant | Simulation | Plant |
| Component | Distillate | Result | Data | Result | Data |
| Toluene | 0.585 | 0.425 | 0.455 | 1.058 | 0.914 |
| 1-Octene | 23.605 | 13.496 | 13.357 | 53.426 | 50.924 |
| n-Octane | 3.629 | 1.522 | 1.867 | 9.844 | 8.294 |
| 2-Hexanone | 0.000 | 0.000 | — | 0.000 | — |
| Hexanal | 0.000 | 0.000 | — | 0.000 | — |
| Water | 7.235 | 9.591 | 9.98 | 0.281 | 0.490 |
| Ethanol | 52.707 | 68.763 | 69.119 | 5.339 | 14.178 |
| Flow Rate | 2408.40 | 1798.69 | | 609.71 | |
| (kg/hr) | | | | | |
| Temperature | 70.53 | 28.00 | | 28.00 | |
| (° C.) | | | | | |

TABLE 61
Stripper Column Results for Data Point 5

| | Input | Simulation Result | Plant Data |
|---|---|---|---|
| Component | Feed | Bottoms | Bottoms |
| Toluene | 0.917 | 1.028 | 0.977 |
| 1-Octene | 47.481 | 58.193 | 59.856 |
| n-Octane | 7.766 | 9.662 | 9.934 |
| 2-Hexanone | 0.000 | 0.000 | — |
| Hexanal | 0.000 | 0.000 | — |
| Water | 0.700 | 0.000 | n.a. |
| Ethanol | 16.977 | 22.7 ppm | 18.9 ppm |
| Flow Rate (kg/hr) | 749.3 | 521.4 | 474 (equivalent) |
| Temperature (° C.) | 50 | 114.00 | 114.3 |

| | |
|---|---|
| Theoretical Stages | 8 |

No converging result for the stripper column distillate phase separation.

Symbol: ‘-’, Status: undetected components on GC results

Symbol: ‘n.a.’, Status: no analysis done

Symbol: ‘*’, Status: Sample re-analysed 2 months later due to misleading analytical results. This analysis was also done on an FFAP column, but with N₂ carrier gas. The 2-Hexanone and 1-Hexanal components are not as easily separated. Use these results as an indication of stream composition.

1. A process for the reduction of oxygenates, including acid, in an olefin and paraffin containing hydrocarbon feed stream, said process including azeotropic distillation of the feed stream using a binary entrainer to recover at least the olefin and paraffin portion of the feed stream.

2. A process as claimed in claim 1, in which the binary entrainer includes a polar species.

3. A process as claimed in claim 2, wherein the polar species is acetonitrile.

4. A process as claimed in claim 1, wherein the binary entrainer includes a solvent which is also a polar species.

5. A process as claimed in claim 1, wherein the binary entrainer includes water.

6. A process as claimed in claim 1, in which the feed stream is of Fischer Tropsch process origin containing hydrocarbons, such as olefins and/or paraffins and/or aromatics, and impurities, such as acid and other oxygenates.

7. A process as claimed in claim 6, in which the feed stream includes C₇ to C₁₂ hydrocarbons of olefinic and paraffinic nature.

8. A process as claimed in clam 1, in which the feed stream is fed to the azeotropic distillation column at an intermediate feed point.

9. A process as claimed in claim 8, wherein the azeotropic disitillation column reflux is a recycle stream that contains a mixture of binary entrainer and olefin enriched hydrocarbons.

10. A process as claimed in claim 4, wherein the binary entrainer is a mixture of ethanol and water.

11. A process as claimed in claim 4, wherein the solvents of the binary entrainer include one or more of methanol, propanol, iso-propanol, butanol, and acetonitrile.
